(19) United States
(12) Patent Application Publication
(10) Pub. No.: US 2016/0010153 A1
(43) Pub. Date: Jan. 14, 2016

(54) NOVEL COMPOSITIONS, METHODS AND KITS FOR BLOOD TYPING

(71) Applicant: LIFE TECHNOLOGIES CORPORATION, Carlsbad, CA (US)

(72) Inventors: Marion LAIG, San Mateo, CA (US); Elena GRIGORENKO, Madison, AL (US)

(73) Assignee: LIFE TECHNOLOGIES CORPORATION, Carlsbad, CA (US)

(21) Appl. No.: 14/772,326

(22) PCT Filed: Mar. 17, 2014
(86) PCT No.: PCT/US14/30706

§ 371 Date: Sep. 2, 2015

Related U.S. Application Data
(60) Provisional application No. 61/852,355, filed on Mar. 15, 2013.

(51) Int. Cl. C12Q 1/68

(52) U.S. Cl. CPC C12Q 1/6881; C12Q 2600/16; C12Q 2600/166; C12Q 2600/158

(57) ABSTRACT

The present disclosure provides compositions, methods, and kits for typing of blood groups by their genotype. In some embodiments, the compositions, methods, and kits include one or more genotype reference nucleic acids.

### RELATED APPLICATIONS

This application claims the benefit under 35 U.S.C. 119(e) of U.S. Provisional Patent Application No. 61/852,355, filed Mar. 15, 2013, which is hereby incorporated by reference in its entirety.

### FIELD

This disclosure generally relates to the field of identification of blood group antigens on erythrocytes, for example, through determining a blood group locus genotype.

### BACKGROUND

Blood transfusions have been recognized as a form of medical intervention for centuries. However, early attempts at transfusions were hindered by the lack of understanding about the existence of and difference between blood types. As a result, many of these early attempts at transfusion were injurious if not fatal to the recipient.

Blood group antigens are markers on the cell surface of erythrocytes, commonly known as red blood cells (RBCs). These surface markers can cause severe immune reactions when the wrong blood type is transfused into a patient, as the recipient's immune system can be triggered by these antigens if they are not recognized. For example, the ABO blood type antigens are based on sugars on the surface of the RBCs and the Rh blood types are based on the presence or absence of a surface protein.

To ensure safe and compatible transfusions, identifying the relevant antigens of RBCs in a blood sample is important. The traditional methods of determining blood typing involve reacting the donor's and recipient's blood samples with surface antigen-specific antibodies, and observing whether agglutination occurs. This traditional method has certain drawbacks, as it requires specific antibodies to each antigen to be tested, and that the antibodies be sufficiently specific. However, blood has been shown to have a multiplicity of different antigens beyond the classical ABO types, which can also affect transfusion compatibility.

Typing is also important when trying match donors to recipients with rare blood types. In such cases, where the expected recipient has a blood type that is not among one of the major blood groups, it is important to identify a suitable donor having a compatible blood type. In other situations, the presence of certain minor blood groups that are difficult to type can nonetheless be incompatible with the donor.

Blood typing remains important for many other clinical applications, such as determining compatibility between an organ donor and recipient. Also, fetal-maternal blood type incompatibility can also result in adverse events if not detected and managed in time.

It is therefore desirable to develop methods, compositions and kits to improve the extent and accuracy of blood typing.

### SUMMARY

Provided herein are compositions, methods and kits for the identification of blood group antigens on erythrocytes.

In one aspect, provided herein are methods for genotyping a nucleic acid sample to determine blood group marker genotypes of the sample. In some embodiments, a method is provided for genotyping a nucleic acid sample which includes performing an amplification reaction on the nucleic acid sample with a plurality of amplification primer pairs to form a plurality of amplification products, wherein each amplification primer pair is specific for at least a portion of a sequence of a blood group locus, wherein the blood groups are selected from the group consisting of: ABO, Colton, Cromer, Diego, Dombrock, Duffy, hemoglobin S, Kell, Kidd, Knops, Landsteiner-Wiener, Lutheran, MNS, RH(D), RH(CE), Scianna, and Yt; and determining the genotype of the amplification products.

In some embodiments of the method, the blood group loci to be amplified include at least one locus of each of the Colton, Cromer, Diego, Dombrock, Duffy, Kell, Kidd, Knops, Landsteiner-Wiener, Lutheran, MNS, Scianna, and Yt blood groups. In some embodiments of the method, the blood group loci to be amplified include at least one locus of each of the Colton, Cromer, Diego, Dombrock, Duffy, Kell, Kidd, Knops, Landsteiner-Wiener, Lutheran, MNS, Scianna, Yt and Rh blood groups. In some embodiments of the method, the blood group loci to be amplified include at least one allele of each of the Rh-RHCE and Rh-RHCD of the Rh blood group.

In some embodiments, a method is provided for genotyping a nucleic acid sample which includes performing a plurality of individual amplification reactions on the nucleic acid sample, where each amplification reaction includes a pair of amplification primers specific for at least portions of a blood group locus sequence that flank at least one marker allele for a group of blood group loci consisting at least of: Colton, Cromer, Diego, Dombrock, Duffy, Kell, Kidd, Knops, Landsteiner-Wiener, Lutheran, MNS, Scianna, and Yt. In some embodiments, the blood group loci amplified also include the Rh blood group. Performing the amplification reactions is followed by determining the genotype of the blood group loci corresponding to the amplification products.

In some embodiments, a high throughput method is provided for genotyping a nucleic acid sample which includes performing multiple parallel amplification reactions on the nucleic acid sample, where each amplification reaction includes a pair of amplification primers specific for at least portions of a blood group locus sequence that flank at least one marker allele for a group of blood group loci consisting at least of: Colton, Cromer, Diego, Dombrock, Duffy, Kell, Kidd, Knops, Landsteiner-Wiener, Lutheran, MNS, Scianna, and Yt. In some embodiments, the blood group loci amplified also include the Rh blood group. Performing the multiple parallel amplification reactions is followed by determining the genotype of the blood group loci corresponding to the amplification products.

In some embodiments, the amplification reactions also include at least one probe specific for a marker allele of the blood group locus amplified by the amplification primer pair.

In some embodiments, the determining step of the method includes determining at least a portion of the nucleic acid sequence of at least one of the loci. In some embodiments, the determining step of the method includes determining the copy number of the nucleic acid sequence of at least one of the loci.

In another aspect, provided herein are kits for blood group genotyping of a nucleic acid sample. In some embodiments, a kit is provided for genotyping a nucleic sample, the kit including a plurality of amplification primer pairs; wherein each amplification primer pair is configured to amplify at least a portion of a sequence of a blood group locus under amplification conditions, wherein the blood groups are selected from the group consisting of: ABO, Colton, Cromer, Diego, Dombrock, Duffy, hemoglobin S, Kell, Kidd, Knops, Landsteiner-Wiener, Lutheran, MNS, Rh, Scianna, and Yt.

In some embodiments of the kit, the blood group loci to be amplified include at least one locus of each of the Colton, Cromer, Diego, Dombrock, Duffy, Kell, Kidd, Knops, Landsteiner-Wiener, Lutheran, MNS, Scianna and Yt blood groups. In some embodiments of the kit, the blood group loci to be amplified include at least one locus of each of the Colton, Cromer, Diego, Dombrock, Duffy, Kell, Kidd, Knops, Landsteiner-Wiener, Lutheran, MNS, Scianna, Yt and Rh blood groups.

In some embodiments, the kit includes at least one amplification primer pair configured to determine the copy number of a blood group locus. In some embodiments, the kit further includes a substrate having a plurality of sites, wherein each site contains one of the amplification primer pairs.

In another aspect, provided herein are compositions including a reference nucleic acid molecule for use in blood group genotyping. In some embodiments, the sequence of the genotyping reference nucleic acid molecule comprises sequences of two or more blood group alleles. In some embodiments, the sequence of the genotyping reference nucleic acid molecule comprises sequences of two or more blood group alleles, where each of the alleles is of a different blood group locus marker. In some embodiments, the genotyping reference includes at least two nucleic acid molecules, where each of the at least two molecules comprises a sequence of at least two different blood group alleles and each of the at least two molecules contains only one allele sequence of a blood group locus marker.

In some embodiments, the reference nucleic acid molecule contains an allele of a blood group locus selected from the group consisting of ABO, Colton, Cromer, Diego, Dombrock, Duffy, hemoglobin S, Kell, Kidd, Knops, Landsteiner-Wiener, Lutheran, MNS, Rh, Scianna and Yt blood group.

In some embodiments, a kit is provided for testing a genotyping assay including a nucleic acid having at least one control nucleic acid sequence, wherein each control nucleic acid sequence is at least a portion of a sequence of a blood group allele.

These and other features of the present teachings are provided herein.

### BRIEF DESCRIPTION OF THE DRAWINGS

The skilled artisan will understand that the drawings described below, are for illustration purposes only. The drawings are not intended to limit the scope of the present teachings in any way.

FIG. 1 is a schematic representation a blood donation qualification process.

FIG. 2 depicts exemplary nucleic acid plasmid reference according to various embodiments described herein.

FIG. 3 is a chart depicting exemplary genotype calls of the Kell Js(a/b) blood group antigen according to various embodiments described herein.

FIG. 4 illustrates exemplary Kell Js(a/b) blood group antigen phenotype calls for the samples of FIG. 3.

### DETAILED DESCRIPTION

To provide a more thorough understanding of the present invention, the following description sets forth numerous specific details, such as specific configurations, parameters, examples, and the like. It should be recognized, however, that such description is not intended as a limitation on the scope of the present invention, but is intended to provide a better description of the exemplary embodiments.

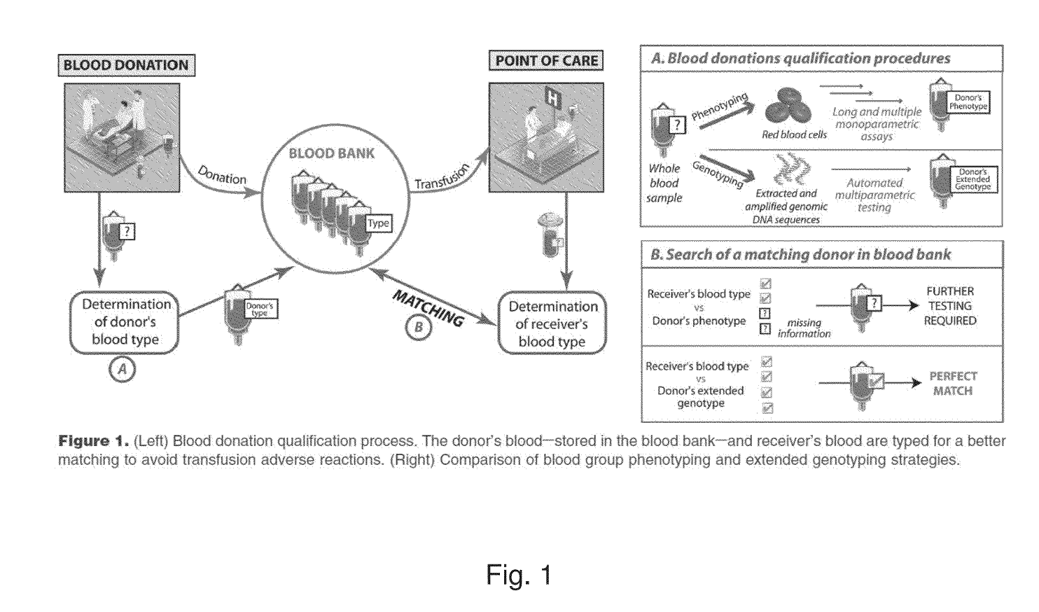

As mentioned above, quick and accurate blood typing is important for safe blood transfusions. An example of a blood transfusion process is illustrated in FIG. 1. The donor's blood is stored in the blood bank. The receiver of the blood and the donor blood are typed to avoid adverse transfusion reactions. Furthermore, FIG. 1 also illustrates a comparison of blood group phenotyping and genotyping strategies.

Blood groups can be determined by analyzing a person's DNA to determine variants in a group of genes known to be important in determining blood type. Typing the blood begins with a blood sample. The blood sample can then be processed according to known methods to extract the DNA from the blood cells. The DNA, a polynucleotide chain, is analyzed according to known methods, such as polymerase chain reaction (PCR).

In general, amplification of a target DNA strand by PCR proceeds through a series of temperature regulated cycles using the activity of a thermostable enzyme and a sequence specific primer set. At an appropriate temperature, primers hybridize to portions of the DNA strand and the enzyme successively adds a plurality of nucleotide bases to elongate the primer resulting in the production of progeny (daughter) strands. Each progeny strand possesses a complimentary composition relative to the target strand from which it was derived and can serve as a target in subsequent reaction cycles.

When applying quantitative methods to PCR-based technologies, a fluorescent probe or other detectable reporter construct may be incorporated into the reaction to provide a means for determining the progress of the target amplification. In the case of a fluorescent probe, the reaction can be made to fluoresce in relative proportion to the quantity of nucleic acid product produced. As such, using PCR, assays for nucleotides sequences corresponding to the blood typing genes and gene variants are the target sequences and are used to determine the blood type of the blood sample.

For example, described in this application, specific assays were designed for Kell Jsa and Jsb alleles based on distinguishing each of the alleles with an allele-specific probe. Further, other assays to determine blood typing genes and gene variants of DNA have been described in published scientific literature. Provided herein are compositions, methods and kits for the detection and genotyping of target nucleic acids.

In some embodiments, the method provided for genotyping a nucleic acid sample includes subjecting the sample to multiple individual amplification reactions, each reaction performed with a pair of amplification primers designed to be specific for at least a portion of a blood group locus. The multiple individual amplification reactions generate individual amplification products for each of the blood group loci for which the amplification primers were designed. The overall blood group genotype of the sample is arrived at by determining the genotype of the amplification products from the individual amplification reactions.

The pair of amplification primers in the individual amplification reactions are specific for at least portions of a blood group locus sequence that flank at least one marker allele. In some embodiments, the amplification reactions generate amplification products from a group of blood group loci consisting at least of: Colton, Cromer, Diego, Dombrock, Duffy, Kell, Kidd, Knops, Landsteiner-Wiener, Lutheran, MNS, Scianna, and Yt. In addition to these loci, in certain embodiments, amplification products are also generated from ABO and/or Rh blood group loci. In other embodiments, the amplification reactions generate amplification products from a group of Rh blood group loci consisting at least of RHCE and RHD loci.

In some embodiments, the amplification reactions generate amplification products from a set of blood group loci selected from the group consisting of ABO, Colton, Cromer, Diego, Dombrock, Duffy, Kell, Kidd, Knops, Landsteiner-Wiener, Lutheran, MNS, Rh, Scianna, and Yt. In certain embodiments, the set is at least 5 of blood group loci selected from the group consisting of ABO, Colton, Cromer, Diego, Dombrock, Duffy, Kell, Kidd, Knops, Landsteiner-Wiener, Lutheran, MNS, Rh, Scianna, and Yt. In other embodiments, the set is at least 7 of blood group loci selected from the group consisting of ABO, Colton, Cromer, Diego, Dombrock, Duffy, Kell, Kidd, Knops, Landsteiner-Wiener, Lutheran, MNS, Rh, Scianna, and Yt. In still other embodiments, the set is at least 9 of blood group loci selected from the group consisting of ABO, Colton, Cromer, Diego, Dombrock, Duffy, Kell, Kidd, Knops, Landsteiner-Wiener, Lutheran, MNS, Rh, Scianna, and Yt.

In some embodiments, the amplification reactions generate amplification products from a set of prevalent blood group loci selected from the group consisting of ABO, Colton, Cromer, Diego, Dombrock, Duffy, Kell, Kidd, Knops, Landsteiner-Wiener, Lutheran, MNS, Rh, Scianna, and Yt. In certain embodiments, the set is at least 5 prevalent blood group loci selected from the group consisting of ABO, Dombrock, Duffy, Kell, Kidd, MNS, and Rh. In some embodiments, the amplification reactions generate amplification products from a set of rare blood group loci selected from the group consisting of ABO, Colton, Cromer, Diego, Dombrock, Duffy, Kell, Kidd, Knops, Landsteiner-Wiener, Lutheran, MNS, Rh, Scianna, and Yt. In certain embodiments, the set is at least 7 rare blood group loci selected from the group consisting of Colton, Cromer, Diego, Dombrock, Knops, Landsteiner-Wiener, Lutheran, Scianna, and Yt.

In some embodiments, determining the genotype of the nucleic acid sample includes determining at least a portion of the nucleic acid sequence of the amplification product for each analyzed blood group locus. In other embodiments, determining the genotype includes determining copy number of the amplified products for each analyzed blood group locus.

In some embodiments, the individual amplification reactions also include at least one probe specific for an allele of the blood group locus amplified by the amplification primers in the reaction. The probe can be a detectably label oligonucleotide probe, such as, without limitation, a fluorescently-labeled oligonucleotide probe. In certain embodiments, the individual amplification reactions include two probes, each probe specific for one allele of the blood group locus amplified by the amplification primer pair. In some embodiments, the two allele-specific probes are differentially labeled, such as with different detectable labels, so that hybridization of either probe to an amplification product can be detected in the presence of the other labeled probe.

In some embodiments, the multiple individual amplification reactions are performed concurrently. In some embodiments, determining the genotype is performed concurrently for each of the of the amplification products.

In some embodiments, high throughput methods are provided for genotyping a nucleic acid sample in which multiple parallel amplification reactions are performed on a nucleic acid sample to generate amplification products in individual reaction chambers. Each amplification reaction includes a pair of amplification primers specific for at least portions of a blood group locus sequence that flank at least one marker allele. In some embodiments, the amplification reactions generate amplification products from a group of blood group loci consisting at least of: Colton, Cromer, Diego, Dombrock, Duffy, Kell, Kidd, Knops, Landsteiner-Wiener, Lutheran, MNS, Scianna, and Yt. In addition to these loci, in certain embodiments, amplification products are also generated from ABO and/or Rh blood group loci. In some embodiments, the amplification reactions generate amplification products from a set of prevalent blood group loci selected from the group consisting of ABO, Colton, Cromer, Diego, Dombrock, Duffy, Kell, Kidd, Knops, Landsteiner-Wiener, Lutheran, MNS, Rh, Scianna, and Yt. In certain embodiments, the set is at least 5 or at least 7 or at least 9 of the listed blood group loci.

In some embodiments, the amplification reactions occur on a substrate having a plurality of reaction sites and each reaction site contains one pair of amplification primers. In some embodiments, the amplification reactions occur in reaction vessels and each reaction contains one pair of amplification primers. In some embodiments, the reaction vessel further contains at least one allele-specific oligonucleotide probe, the probe being specific for an allele of the blood group locus amplified by the amplification primer pair present in the reaction vessel. In certain embodiments, the reaction vessel contains at least two probes, each probe specific for one allele of the blood group locus amplified by the pair of amplification primers in the reaction vessel. In some embodiments, the two allele-specific probes are differentially labeled, such as with different detectable labels, so that hybridization of either probe to an amplification product can be detected in the presence of the other labeled probe. In certain embodiments, the reaction vessels are through-holes in a substrate plate and each through-hole contains one pair of amplication primers and at least one detectably-labeled probe as described herein.

The amplification reaction vessel can also contain other component reagents of the amplification reaction mixture such as, for example, dNTPs (dATP, dCTP, dGTP and/or dTTP), polymerase, buffer(s), and/or salt(s).

The methods, compositions, and kits provided herein are of use in activities for which determination of a sample's blood group phenotype is desired. For example, the methods, compositions, and kits are for use in screening, characterizing, and/or segregating a blood bank's collected blood specimens. In some embodiments, the provided methods, compositions, and kits are for use in confirming a blood group type of a blood sample, for example, confirming a sample's blood group type which had previously been deemed ambiguous. In some embodiments, the provided methods, compositions, and kits are for use in determining suitability of a blood sample for transfusion. In some embodiments, the provided methods, compositions, and kits are for use in selecting a transfusion donor by determining the blood group genotype of a sample from a perspective donor and comparing compatibility of the donor genotype to the genotype of the transfusion recipient.

In some embodiments, the markers include one or more markers corresponding to alleles that determine the phenotypes of the ABO blood group system. The locus encodes glycosyltransferases that determine the carbohydrate antigens on band 3 protein or glycosphingolipid. In some embodiments, the markers correspond to ABO alleles A, B or O (negative phenotype). In some embodiments, the markers correspond to other mutations in the ABO coding or regulatory sequences.

In some embodiments, the markers include one or more markers corresponding to alleles encoding phenotypes of the Colton blood group system or Co antigen (also known as aquaporin-1), a membrane marker found on RBCs and kidney tubules. In some embodiments, the markers correspond to Colton alleles Co(a), Co(b) or Co-negative. In some embodiments, the markers correspond to other mutations in the Colton coding or regulatory sequences.

In some embodiments, the markers include one or more markers corresponding to alleles encoding phenotypes of the Cromer blood group system or CROM antigen (also known as decay-accelerating factor or DAF), a membrane marker that regulate complement activity found on RBCs. In some embodiments, the markers correspond to Cromer antigen Cra, SERF, ZENA or Inab (a Cromer negative phenotype). In some embodiments, the markers correspond to other mutations in the Cromer coding or regulatory sequences.

In some embodiments, the markers include one or more markers corresponding to alleles encoding phenotypes of the Diego (or Wright) blood group system (also known as band 3 protein, SLC4A1), a chloride/carbonate channel found on RBCs. In some embodiments, the markers correspond to Diego alleles Diegoa (Dia− and Dia+), Diegob (Dib− and Dib+), Wra and Wrb. In some embodiments, the markers correspond to other mutations in the Diego coding or regulatory sequences.

In some embodiments, the markers include one or more markers corresponding to alleles encoding phenotypes of the Dombrock or Do blood group system (also known as ART4 or CD297), a glycoprotein member of the ADP-ribosyltransferase gene family found on RBCs. In some embodiments, the markers correspond to Dombrock alleles Doa and Dob, Gya, Hy or Joa. In some embodiments, the markers correspond to other mutations in the Dombrock coding or regulatory sequences.

In some embodiments, the markers include one or more markers corresponding to alleles encoding phenotypes of the Duffy blood group system (also known as Fy glycoprotein or CD234), a surface marker found on RBCs. In some embodiments, the markers correspond to Duffy alleles FY*A, FY*B, FY*X and FY*Fy that encode for Fy-a antigen (G1y42), Fy-b antigen (Asp42), Fy-x phenotype (Cys89 and Thr100; or Ser49) and Fy-y antigens, respectively. In some embodiments, the markers correspond to a negative phenotype Fy-o, such as the Fy GATA promoter silencing mutation. In some embodiments, the markers correspond to other mutations in the Duffy coding or regulatory sequences.

In some embodiments, the markers include one or more markers corresponding to alleles encoding hemoglobin S or HbS (also known as sickle-cell hemoglobin).

In some embodiments, the markers include one or more markers corresponding to alleles encoding phenotypes of the Kell or K or Kp blood group system, a transmembrane zinc-dependent endopeptidase on RBCs. In some embodiments, the markers correspond to Kell antigens K1 (Kell), K2 (k or Cellano), Kpa, Kpb, Jsa, Jsb, and K null. In some embodiments, the markers correspond to other mutations in the Kell coding or regulatory sequences.

In some embodiments, the markers include one or more markers corresponding to alleles encoding phenotypes of the Kidd blood group system. In some embodiments, the markers correspond to Kidd antigens Jk(a), Jk(b) or Jk (finnish null). In some embodiments, the markers correspond to other mutations in the Kidd coding or regulatory sequences.

In some embodiments, the markers include one or more markers corresponding to alleles encoding phenotypes of the Knops or KN blood group system, a complement receptor 1 glycoprotein (CR1, CD35). In some embodiments, the markers correspond to Knops antigens Kn(a) and Kn(b) and McCa+ or McCb+ or SI1 or SI2. In some embodiments, the markers correspond to other mutations in the Knops coding or regulatory sequences.

In some embodiments, the markers include one or more markers corresponding to alleles encoding phenotypes of the Landsteiner-Wiener or LW blood group system, a membrane glycoprotein (ICAM-4 or CD242) on RBCs. In some embodiments, the markers correspond to LW(a), LW(b) and LW null. In some embodiments, the markers correspond to other mutations in the LW coding or regulatory sequences.

In some embodiments, the markers include one or more markers corresponding to alleles encoding phenotypes of the Lewis or Le blood group system on RBCs. In some embodiments, the markers correspond to Le(a), Le(b) and Le null. In some embodiments, the markers correspond to other mutations in the Lewis coding or regulatory sequences.

In some embodiments, the markers include one or more markers corresponding to alleles encoding phenotypes of the Lutheran or LU blood group system, an immunoglobulin family member on RBCs. In some embodiments, the markers correspond to Lua and Lub null. In some embodiments, the markers correspond to other mutations in the LU coding or regulatory sequences.

In some embodiments, the markers include one or more markers corresponding to alleles encoding phenotypes of the MNS blood group system, based on glycophorin A (GYPA) and glycophorin B (GYPB) on RBCs. In some embodiments, the markers correspond to MNS antigens M and N (based on GYPA), S and s (based on GYPB), U (high frequency antigen, S- and s-), GYPB silencing +5intron5, GYPB silencing nt230. In some embodiments, the markers correspond to other mutations in the MNS coding or regulatory sequences.

In some embodiments, the markers include one or more markers corresponding to alleles encoding phenotypes of the MNS blood group system, based on glycophorin A (GYPA) and glycophorin B (GYPB) on RBCs. In some embodiments, the markers correspond to MNS antigens M and N (based on GYPA), S and s (based on GYPB), U (high frequency antigen, S- and s-), GYPB silencing +5intron5, GYPB silencing nt230, GYPB silencing nt143. In some embodiments, the markers correspond to other mutations in the MNS coding or regulatory sequences.

In some embodiments, the markers include one or more markers corresponding to alleles encoding phenotypes of the Rhesus or Rh blood group system on RBCs. In some embodiments, the markers correspond to Rh antigens Rh(C), Rh(c), Rh(E), Rh(e), Rh(D), Rh(D deletion), Rh(D 37 bp duplication or RHD-psi), Rh(D hybrid), Rh(C*), Rh(D-CE (4-7)-D hybrid) or null. In some embodiments, the markers correspond to other mutations in the Rh coding or regulatory sequences.

In some embodiments, the markers include one or more markers corresponding to alleles encoding phenotypes of the Scianna or SC blood group system on RBCs. In some embodiments, the markers correspond to Scianna antigens Sc1 and Sc2 or null. In some embodiments, the markers correspond to other mutations in the Scianna coding or regulatory sequences.

In some embodiments, the markers include one or more markers corresponding to alleles encoding phenotypes of the Yt or Cartwright blood group system, an acetylcholinesterase on RBCs. In some embodiments, the markers correspond to Yt antigens Yt(a) and Yt(b) or null. In some embodiments, the markers correspond to other mutations in the Yt coding or regulatory sequences.

To more clearly and concisely describe and point out the subject matter of the present disclosure, the following definitions are provided for specific terms, which are used in the following description and the appended claims. Throughout the specification, exemplification of specific terms should be considered as non-limiting examples.

As used in this specification, the words “a” or “an” means at least one, unless specifically stated otherwise. In this specification, the use of the singular includes the plural unless specifically stated otherwise. For example, but not as a limitation, “a target nucleic acid” means that more than one target nucleic acid can be present; for example, one or more copies of a particular target nucleic acid species, as well as two or more different species of target nucleic acid. The term “and/or” means that the terms before and after the slash can be taken together or separately. For illustration purposes, but not as a limitation, “X and/or Y” can mean “X” or “Y” or “X” and “Y”.

It will be appreciated that there is an implied “about” prior to the temperatures, concentrations, times, etc. discussed in the present disclosure, such that slight and insubstantial deviations are within the scope of the present teachings herein. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the above specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. All literature cited in the specification, including but not limited to, patent, patent applications, articles, books and treatises are expressly incorporated by reference in their entirety for any purpose. In the event that any of the incorporated literature contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The terms “amplicon” and “amplification product” as used herein generally refer to the product of an amplification reaction. An amplicon may be double-stranded or single-stranded, and may include the separated component strands obtained by denaturing a double-stranded amplification product. In certain embodiments, the amplicon of one amplification cycle can serve as a template in a subsequent amplification cycle.

The terms “annealing” and “hybridizing”, including, without limitation, variations of the root words “hybridize” and “anneal”, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability. Conditions under which primers and probes anneal to complementary sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968).

In general, whether such annealing takes place is influenced by, among other things, the length of the complementary portions of the complementary portions of the primers and their corresponding binding sites in the target flanking sequences and/or amplicons, or the corresponding complementary portions of a reporter probe and its binding site; the pH; the temperature; the presence of mono- and divalent cations; the proportion of G and C nucleotides in the hybridizing region; the viscosity of the medium; and the presence of denaturants. Such variables influence the time required for hybridization. Thus, the preferred annealing conditions will depend upon the particular application. Such conditions, however, can be routinely determined by persons of ordinary skill in the art, without undue experimentation. Preferably, annealing conditions are selected to allow the primers and/or probes to selectively hybridize with a complementary sequence in the corresponding target flanking sequence or amplicon, but not hybridize to any significant degree to different target nucleic acids or non-target sequences in the reaction composition at the second reaction temperature.

The term “selectively hybridize” and variations thereof, means that, under appropriate stringency conditions, a given sequence (for example, but not limited to, a primer) anneals with a second sequence comprising a complementary string of nucleotides (for example, but not limited to, a target flanking sequence or primer binding site of an amplicon), but does not anneal to undesired sequences, such as non-target nucleic acids, probes, or other primers. Typically, as the reaction temperature increases toward the melting temperature of a particular double-stranded sequence, the relative amount of selective hybridization generally increases and mis-priming generally decreases. In this specification, a statement that one sequence hybridizes or selectively hybridizes with another sequence encompasses situations where the entirety of both of the sequences hybridize or selectively hybridize to one another, and situations where only a portion of one or both of the sequences hybridizes or selectively hybridizes to the entire other sequence or to a portion of the other sequence.

As used herein, the term “stringency” is used to define the temperature and solvent composition existing during hybridization and the subsequent processing steps at which a hybrid comprised of two complementary nucleotide sequences will form. Stringency also defines the amount of homology, the conditions necessary, and the stability of hybrids formed between two nucleotide sequences. As the stringency conditions increase, selective hybridization is favored and non-specific cross-hybridization is disfavored. Increased stringency conditions typically correspond to higher incubation temperature, lower salt concentrations, and/or higher pH, relative to lower stringency conditions at which mis-priming is more likely to occur. Those in the art understand that appropriate stringency conditions to enable the selective hybridization of a primer or primer pair to a corresponding target flanking sequence and/or amplicon can be routinely determined using well known techniques and without undue experimentation (see, e.g., PCR: The Basics from Background to Bench, McPherson and Moller, Bios Scientific Publishers, 2000).

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The terms “denaturing” and “denaturation” as used herein refer to any process in which a double-stranded polynucleotide, including without limitation, a genomic DNA (gDNA) fragment comprising at least one target nucleic acid, a double-stranded amplicon, or a polynucleotide comprising at least one double-stranded segment is converted to two single-stranded polynucleotides or to a single-stranded or substantially single-stranded polynucleotide, as appropriate. Denaturing a double-stranded polynucleotide includes, without limitation, a variety of thermal and chemical techniques which render a double-stranded nucleic acid single-stranded or substantially single-stranded, for example but not limited to, releasing the two individual single-stranded components of a double-stranded polynucleotide or a duplex comprising two oligonucleotides. Those in the art will appreciate that the denaturing technique employed is generally not limiting unless it substantially interferes with a subsequent annealing or enzymatic step of an amplification reaction, or in certain methods, the detection of a fluorescent signal.

As used herein, the term “Tm” is used in reference to melting temperature. The melting temperature is the temperature at which a population of double-stranded nucleic acid molecules becomes half dissociated into single strands.

The term “minor groove binder” as used herein refers to a small molecule that fits into the minor groove of double-stranded DNA, sometimes in a sequence specific manner Generally, minor groove binders are long, flat molecules that can adopt a crescent-like shape and thus, fit snugly into the minor groove of a double helix, often displacing water. Minor groove binding molecules typically comprise several aromatic rings connected by bonds with torsional freedom, for example, but not limited to, furan, benzene, or pyrrole rings.

The term “end-point” measurement refers to a method where data collection occurs only once the reaction has been stopped.

The terms “real-time” and “real-time continuous” are interchangeable and refer to a method where data collection occurs through periodic monitoring during the course of the polymerization reaction. Thus, the methods combine amplification and detection into a single step.

As used herein, the term “quantitative PCR” refers to the use of PCR to quantify gene expression.

As used herein the terms “Cₜ” and “cycle threshold” refer to the time at which fluorescence intensity is greater than background fluorescence. They are characterized by the point in time (or PCR cycle) where the target amplification is first detected. Consequently, the greater the quantity of target DNA in the starting material, the faster a significant increase in fluorescent signal will appear, yielding a lower Cₜ.

As used herein, the term “primer” refers to a synthetically or biologically produced single-stranded oligonucleotide that is extended by covalent bonding of nucleotide monomers during amplification or polymerization of a nucleic acid molecule. Nucleic acid amplification often is based on nucleic acid synthesis by a nucleic acid polymerase or reverse transcriptase. Many such polymerases or reverse transcriptases require the presence of a primer that may be extended to initiate such nucleic acid synthesis. A primer is typically 11 bases or longer; most preferably, a primer is 17 bases or longer, although shorter or longer primers may be used depending on the need. As will be appreciated by those skilled in the art, the oligonucleotides disclosed herein may be used as one or more primers in various extension, synthesis, or amplification reactions.

Typically, a PCR reaction employs a pair of amplification primers including an “upstream” or “forward” primer and a “downstream” or “reverse” primer, which delimit a region of the RNA or DNA to be amplified. A first primer and a second primer may be either a forward or reverse primer and are used interchangeably herein and are not to be limiting.

The terms “complementarity” and “complementary” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementarity refers to the situation in which each nucleotide unit of one polynucleotide strand or region can hydrogen bond with each nucleotide unit of a second polynucleotide strand or region. “Less than perfect complementarity” refers to the situation in which some, but not all, nucleotide units of two strands or two units can hydrogen bond with each other.

As used herein, the term “reverse complement” refers to a sequence that will anneal/base pair or substantially anneal/base pair to a second oligonucleotide according to the rules defined by Watson-Crick base pairing and the antiparallel nature of the DNA-DNA, RNA-RNA, and RNA-DNA double helices. Thus, as an example, the reverse complement of the RNA sequence 5′-AAUUUGC would be 5′-GCAAAUU. Alternative base pairing schemes, including but not limited to G-U pairing, can also be included in reverse complements.

As used herein, the term “probe” refers to synthetic or biologically produced nucleic acids (DNA or RNA) which, by design or selection, contain specific nucleotide sequences that allow them to hybridize, under defined stringencies, specifically (i.e., preferentially) to target nucleic acid sequences.

As used herein, “substantially less extendable” is used to characterize an oligonucleotide that is inefficiently extended or not extended in an extension and/or amplification reaction when the 3′ most nucleotide of the oligonucleotide is not complementary to the corresponding base of a target/template nucleic acid.

As used herein, the term “template” is interchangeable with “target molecule” or “target nucleic acid” and refers to a double-stranded or single-stranded nucleic acid molecule which is to be amplified, copied or extended, synthesized, or sequenced. In the case of a double-stranded DNA molecule, denaturation of its strands to form a first and a second strand is performed to amplify, sequence, or synthesize these molecules. A primer, complementary to a portion of a template is hybridized under appropriate conditions and the polymerase (DNA polymerase or reverse transcriptase) may then synthesize a nucleic acid molecule complementary to said template or a portion thereof. The newly synthesized molecule, according to the present disclosure, may be equal or shorter in length than the original template. Mismatch incorporation during the synthesis or extension of the newly synthesized molecule may result in one or a number of mismatched base pairs. Thus, the synthesized molecule need not be exactly complementary to the template. The template may be an RNA molecule, a DNA molecule, or a DNA/RNA hybrid molecule. A newly synthesized molecule may serve as a template for subsequent nucleic acid synthesis or amplification.

The target nucleic acid may be obtained from any source, and may comprise any number of different compositional components. For example, the target may be a nucleic acid (e.g., DNA or RNA), transfer RNA (tRNA), small interfering RNA (siRNA), microRNA (miRNA), or other mature small RNA, and may comprise nucleic acid analogs or other nucleic acid mimics The target may be methylated, non-methylated, or both. The target may be bisulfite-treated and non-methylated cytosines converted to uracil. Further, it will be appreciated that “target nucleic acid” may refer to the target nucleic acid itself, as well as surrogates thereof, for example, amplification products and native sequences. The target molecules of the present teachings may be derived from any number of sources, including without limitation, viruses, archae, protists, prokaryotes and eukaryotes, for example, but not limited to, plants, fungi, and animals. These sources may include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil), research samples generally, purified samples generally, cultured cells and lysed cells. It will be appreciated that target nucleic acids may be isolated from samples using any of a variety of procedures known in the art, for example, the Applied Biosystems ABI Prism® 6100 Nucleic Acid PrepStation (Life Technologies, Foster City, Calif.) and the ABI Prism® 6700 Automated Nucleic Acid Workstation (Life Technologies, Foster City, Calif.), Ambion® mirVana™ RNA isolation kit (Life Technologies, Austin, Tex.), and the like. It will be appreciated that target nucleic acids may be cut or sheared prior to analysis, including the use of such procedures as mechanical force, sonication, restriction endonuclease cleavage, or any method known in the art. In general, the target nucleic acids of the present teachings will be single-stranded, though in some embodiments the target nucleic acids may be double-stranded, and a single-strand may result from denaturation.

As used herein, the terms “hairpin” and “stem-loop” are interchangeable and are used to indicate the structure of an oligonucleotide in which one or more portions of the oligonucleotide form base pairs with one or more other portions of the oligonucleotide. When the two portions are base paired to form a double-stranded portion of the oligonucleotide, the double-stranded portion may be referred to as a stem. Thus, depending on the number of complementary portions used, a number of stems (preferably about 1 to about 10) may be formed.

The term “incorporating” as used herein, means becoming a part of a DNA or RNA molecule or primer.

The term “nucleic acid binding dye” as used herein refers to a fluorescent molecule that is specific for a double-stranded polynucleotide or that at least shows a substantially greater fluorescent enhancement when associated with double-stranded polynucleotides than with a single stranded polynucleotide. Typically, nucleic acid binding dye molecules associate with double-stranded segments of polynucleotides by intercalating between the base pairs of the double-stranded segment, but binding in the major or minor grooves of the double-stranded segment, or both. Non-limiting examples of nucleic acid binding dyes include ethidium bromide, DAPI, Hoechst derivatives including without limitation Hoechst 33258 and Hoechst 33342, intercalators comprising a lanthanide chelate (for example, but not limited to, a naphthalene diimide derivative carrying two fluorescent tetradentate β-diketone-Eu³⁺ chelates (NDI-(BHHCT-Eu³⁺)₂), see e.g., Nojima et al., Nucl. Acids Res. Suppl. No. 1 105 (2001), and certain unsymmetrical cyanine dyes such as SYBR® Green and PicoGreen®.

As used herein, the terms “polynucleotide”, “oligonucleotide,” and “nucleic acid” are used interchangeably and refer to single-stranded and double-stranded polymers of nucleotide monomers, including without limitation, 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺, and the like. A polynucleotide may be composed entirely of deoxyribonucleotides, entirely of ribonucleotides, or chimeric mixtures thereof and may include nucleotide analogs. The nucleotide monomer units may comprise any of the nucleotides described herein, including, but not limited to, nucleotides and/or nucleotide analogs. Polynucleotides typically range in size from a few monomeric units, e.g., 5-40 when they are sometimes referred to in the art as oligonucleotides, to several thousands of monomeric nucleotide units. Unless denoted otherwise, whenever a polynucleotide sequence is represented, it will be understood that the nucleotides are in the 5′-to-3′ order from left to right and that “A” denotes deoxyadenosine, “C” denotes deoxycytosine, “G” denotes deoxyguanosine, “T” denotes deoxythymidine, and “U” denotes deoxyuridine, unless otherwise noted.

The term “nucleotide” refers to a phosphate ester of a nucleoside, e.g., triphosphate esters, wherein the most common site of esterification is the hydroxyl group attached at the C-5 position of the pentose.

The term “nucleoside” refers to a compound consisting of a purine, deazapurine, or pyrimidine nucleoside base, e.g., adenine, guanine, cytosine, uracil, thymine, deazaadenine, deazaguanosine, and the like, linked to a pentose at the 1′ position, including 2′-deoxy and 2′-hydroxyl forms. When the nucleoside base is purine or 7-deazapurine, the pentose is attached to the nucleobase at the 9-position of the purine or deazapurine, and when the nucleobase is purimidine, the pentose is attached to the nucleobase at the 1-position of the pyrimidine.

The term “analog” includes synthetic analogs having modified base moieties, modified sugar moieties, and/or modified phosphate ester moieties. Phosphate analogs generally comprise analogs of phosphate wherein the phosphorous atom is in the +5 oxidation state and one or more of the oxygen atoms is replaced with a non-oxygen moiety, e.g. sulfur. Exemplary phosphate analogs include: phosphorothioate, phosphorodithioate, phosphoroselenoate, phosphorodiselenoate, phosphoroanilothioate, phosphoranilidate, phosphoramidate, boronophosphates, including associated counterions, e.g., H⁺, NH₄⁺, Na⁺. Exemplary base analogs include: 2,6-diaminopurine, hypoxanthine, pseudouridine, C-5-propyne, isocytosine, isoguanine, 2-thiopyrimidine. Exemplary sugar analogs include: 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, and bromo.

As used herein, the term “reaction vessel” generally refers to any container, chamber, device, or assembly, in which a reaction can occur in accordance with the present teachings. In some embodiments, a reaction vessel may be a microtube, for example, but not limited to, a 0.2 mL or a 0.5 mL reaction tube such as a MicroAmp® Optical tube (Life Technologies Corp., Carslbad, Calif.) or a micro-centrifuge tube, or other containers of the sort in common practice in molecular biology laboratories. In some embodiments, a reaction vessel comprises a well of a multi-well plate (such as a 48-, 96-, or 384-well microtiter plate), a spot on a glass slide, or a channel or chamber of a microfluidics device, including without limitation a TaqMan® Low Density Array or a TaqMan® OpenArray® Real-Time PCR plate (both from Life Technologies Corp.). For example, but not as a limitation, a plurality of reaction vessels can reside on the same support. An OpenArray® Plate, for example, is a reaction plate 3072 through-holes. Each such through-hole in such a plate may contain a single genotyping assay. In some embodiments, lab-on-a-chip-like devices available, for example, from Caliper, Fluidigm and Life Technologies Corp., including the Ion 316™ and Ion 318™ Chip, may serve as reaction vessels in the disclosed methods. It will be recognized that a variety of reaction vessels are commercially available or can be designed for use in the context of the present teachings.

The term “reporter group” is used in a broad sense herein and refers to any identifiable tag, label, or moiety.

The term “thermostable” when used in reference to an enzyme, refers to an enzyme (such as a polypeptide having nucleic acid polymerase activity) that is resistant to inactivation by heat. A “thermostable” enzyme is in contrast to a “thermolabile” polymerase, which can be inactivated by heat treatment. Thermolabile proteins can be inactivated at physiological temperatures, and can be categorized as mesothermostable (inactivation at about 45° C. to about 65° C.), and thermostable (inactivation at greater than about 65° C.). For example, the activities of the thermolabile T5 and T7 DNA polymerases can be totally inactivated by exposing the enzymes to a temperature of about 90° C. for about 30 seconds. A thermostable polymerase activity is more resistant to heat inactivation than a thermolabile polymerase. However, a thermostable polymerase does not mean to refer to an enzyme that is totally resistant to heat inactivation; thus heat treatment may reduce the polymerase activity to some extent. A thermostable polymerase typically will also have a higher optimum temperature than thermolabile DNA polymerases.

The term “working concentration” refers to the concentration of a reagent that is at or near the optimal concentration used in a solution to perform a particular function (such as amplification or digestion of a nucleic acid molecule). The working concentration of a reagent is also described equivalently as a “1× concentration” or a “1× solution” (if the reagent is in solution) of the reagent. Accordingly, higher concentrations of the reagent may also be described based on the working concentration; for example, a “2× concentration” or a “2× solution” of a reagent is defined as a concentration or solution that is twice as high as the working concentration of the reagent; a “5× concentration” or a “5× solution” is five times as high as the working concentration, and so on.

As used herein, the terms “amplification”, “nucleic acid amplification”, or “amplifying” refer to the production of multiple copies of a nucleic acid template, or the production of multiple nucleic acid sequence copies that are complementary to the nucleic acid template. The terms (including the term “polymerizing”) may also refer to extending a nucleic acid template (e.g., by polymerization). The amplification reaction may be a polymerase-mediated extension reaction such as, for example, a polymerase chain reaction (PCR). However, any of the known amplification reactions may be suitable for use as described herein. The term “amplifying” that typically refers to an “exponential” increase in target nucleic acid may be used herein to describe both linear and exponential increases in the numbers of a select target sequence of nucleic acid.

The term “amplification reaction mixture” and/or “master mix” may refer to an aqueous solution comprising the various (some or all) reagents used to amplify a target nucleic acid. Such reactions may also be performed using solid supports (e.g., an array). The reactions may also be performed in single or multiplex format as desired by the user. These reactions typically include enzymes, aqueous buffers, salts, amplification primers, target nucleic acid, and nucleoside triphosphates. Depending upon the context, the mixture can be either a complete or incomplete amplification reaction mixture. The method used to amplify the target nucleic acid may be any available to one of skill in the art. Any in vitro means for multiplying the copies of a target sequence of nucleic acid may be utilized. These include linear, logarithmic, and/or any other amplification method. While this disclosure may generally discuss PCR as the nucleic acid amplification reaction, it is expected that the modified detergents describe herein should be effective in other types of nucleic acid amplification reactions, including both polymerase-mediated amplification reactions (such as helicase-dependent amplification (HDA), recombinase-polymerase amplification (RPA), and rolling circle amplification (RCA)), as well as ligase-mediated amplification reactions (such as ligase detection reaction (LDR), ligase chain reaction (LCR), and gap-versions of each), and combinations of nucleic acid amplification reactions such as LDR and PCR (see, for example, U.S. Pat. No. 6,797,470). For example, the modified detergents may be used in, for example, various ligation-mediated reactions, where for example ligation probes are employed as opposed to PCR primers. Additional exemplary methods include polymerase chain reaction (PCR; see, e.g., U.S. Pat. Nos. 4,683,202; 4,683,195; 4,965,188; and/or 5,035,996), isothermal procedures (using one or more RNA polymerases (see, e.g., PCT Publication No. WO 2006/081222), strand displacement (see, e.g., U.S. Pat. No. RE39007E), partial destruction of primer molecules (see, e.g., PCT Publication No. WO 2006/087574)), ligase chain reaction (LCR) (see, e.g., Wu, et al., Genomics 4: 560-569 (1990)), and/or Barany, et al. Proc. Natl. Acad. Sci. USA 88:189-193 (1991)), Qβ RNA replicase systems (see, e.g., PCT Publication No. WO 1994/016108), RNA transcription-based systems (e.g., TAS, 3SR), rolling circle amplification (RCA) (see, e.g., U.S. Pat. No. 5,854,033; U.S. Patent Application Publication No. 2004/265897; Lizardi et al. Nat. Genet. 19: 225-232 (1998); and/or Barter et al. Nucleic Acid Res., 26: 5073-5078 (1998)), and strand displacement amplification (SDA) (Little, et al. Clin. Chem. 45:777-784 (1999)), among others. These systems, along with the many other systems available to the skilled artisan, may be suitable for use in polymerizing and/or amplifying target nucleic acids for use as described herein.

“Amplification efficiency” may refer to any product that may be quantified to determine copy number (e.g., the term may refer to a PCR amplicon, an LCR ligation product, and/or similar product). The amplification and/or polymerization efficiency may be determined by various methods known in the art, including, but not limited to, determination of calibration dilution curves and slope calculation, determination using qBase software as described in Hellemans et al., Genome Biology 8:R19 (2007), determination using the delta delta Cq (ΔΔCq) calculation as described by Livak and Schmittgen, Methods 25:402 (2001), or by the method as described by Pfaffl, Nucl. Acids Res. 29:e45 (2001), all of which are herein incorporated by reference in their entirety.

In certain embodiments, amplification techniques comprise at least one cycle of amplification, for example, but not limited to, the steps of: denaturing a double-stranded nucleic acid to separate the component strands; hybridizing a primer to a target flanking sequence or a primer-binding site of an amplicon (or complements of either, as appropriate); and synthesizing a strand of nucleotides in a template-dependent manner using a DNA polymerase. The cycle may or may not be repeated. In certain embodiments, a cycle of amplification comprises a multiplicity of amplification cycles, for example, but not limited to 20 cycles, 25 cycles, 30 cycles, 35 cycles, 40 cycles, 45 cycles or more than 45 cycles of amplification.

In some embodiments, amplifying comprises thermocycling using an instrument, for example, but not limited to, a GeneAmp® PCR System 9700, 9600, 2700 or 2400 thermocycler, an Applied Biosystems® ViiA™ 7 Real-Time PCR System, an Applied Biosystems® 7500 Fast Real-Time PCR System, a 7900HT Fast Real-Time PCR System, a StepOne® Real-Time PCR System, a StepOnePlus® Real-Time PCR System, a QuantStudio™ 12K Flex Real-Time PCR System, and the like (all available from Life Technologies Corp., Carlsbad, Calif.). In certain embodiments, single-stranded amplicons are generated in an amplification reaction, for example, but not limited to asymmetric PCR or A-PCR.

In some embodiments, amplification comprises a two-step reaction including without limitation, a pre-amplification step wherein a limited number of cycles of amplification occur (for example, but not limited to, 2, 3, 4, or 5 cycles of amplification), then the resulting amplicon is generally diluted and portions of the diluted amplicon are subjected to additional cycles of amplification in a subsequent amplification step (see, e.g., U.S. Pat. No. 6,605,451 and U.S. Patent Application Publication No. 2004/0175733).

In certain embodiments, an amplification reaction comprises multiplex amplification, in which a multiplicity of different target nucleic acids and/or a multiplicity of different amplification product species are simultaneously amplified using a multiplicity of different primer sets. In certain embodiments, a multiplex amplification reaction and a single-plex amplification reaction, including a multiplicity of single-plex or lower-plexy reactions (for example, but not limited to a two-plex, a three-plex, a four-plex, a five-plex or a six-plex reaction) are performed in parallel.

Exemplary methods for polymerizing and/or amplifying nucleic acids include, for example, polymerase-mediated extension reactions. For instance, the polymerase-mediated extension reaction can be the polymerase chain reaction (PCR). In other embodiments, the nucleic acid amplification reaction is a multiplex reaction. For instance, exemplary methods for polymerizing and/or amplifying and detecting nucleic acids suitable for use as described herein are commercially available as TaqMan® (see, e.g., U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and/or 7,445,900, all of which are hereby incorporated herein by reference in their entirety). TaqMan® assays are typically carried out by performing nucleic acid amplification on a target polynucleotide using a nucleic acid polymerase having 5′-to-3′ nuclease activity, a primer capable of hybridizing to said target polynucleotide, and an oligonucleotide probe capable of hybridizing to said target polynucleotide 3′ relative to said primer. The oligonucleotide probe typically includes a detectable label (e.g., a fluorescent reporter molecule) and a quencher molecule capable of quenching the fluorescence of said reporter molecule. Typically, the detectable label and quencher molecule are part of a single probe. As amplification proceeds, the polymerase digests the probe to separate the detectable label from the quencher molecule. The detectable label (e.g., fluorescence) is monitored during the reaction, where detection of the label corresponds to the occurrence of nucleic acid amplification (e.g., the higher the signal the greater the amount of amplification). Variations of TaqMan® assays (e.g., LNA™ spiked TaqMan® assay) are known in the art and would be suitable for use in the methods described herein.

Another exemplary system suitable for use as described herein utilizes double-stranded probes in displacement hybridization methods (see, e.g., Morrison et al. Anal. Biochem., 18:231-244 (1989); and/or Li, et al. Nucleic Acids Res., 30(2,e5) (2002)). In such methods, the probe typically includes two complementary oligonucleotides of different lengths where one includes a detectable label and the other includes a quencher molecule. When not bound to a target nucleic acid, the quencher suppresses the signal from the detectable label. The probe becomes detectable upon displacement hybridization with a target nucleic acid. Multiple probes may be used, each containing different detectable labels, such that multiple target nucleic acids may be queried in a single reaction.

Additional exemplary methods for polymerizing and/or amplifying and detecting target nucleic acids suitable for use as described herein involve “molecular beacons”, which are single-stranded hairpin shaped oligonucleotide probes. In the presence of the target sequence, the probe unfolds, binds and emits a signal (e.g., fluoresces). A molecular beacon typically includes at least four components: 1) the “loop”, an 18-30 nucleotide region which is complementary to the target sequence; 2) two 5-7 nucleotide “stems” found on either end of the loop and being complementary to one another; 3) at the 5′ end, a detectable label; and 4) at the 3′ end, a quencher moiety that prevents the detectable label from emitting a single when the probe is in the closed loop shape (e.g., not bound to a target nucleic acid). Thus, in the presence of a complementary target, the “stem” portion of the beacon separates out resulting in the probe hybridizing to the target. Other types of molecular beacons are also known and may be suitable for use in the methods described herein. Molecular beacons may be used in a variety of assay systems. One such system is nucleic acid sequence-based amplification (NASBA®), a single step isothermal process for polymerizing and/or amplifying RNA to double stranded DNA without temperature cycling. A NASBA reaction typically requires avian myeloblastosis virus (AMV), reverse transcriptase (RT), T7 RNA polymerase, RNase H, and two oligonucleotide primers. After amplification, the amplified target nucleic acid may be detected using a molecular beacon. Other uses for molecular beacons are known in the art and would be suitable for use in the methods described herein.

The Scorpions™ system is another exemplary assay format that may be used in the methods described herein. Scorpions™ primers are bi-functional molecules in which a primer is covalently linked to the probe, along with a detectable label (e.g., a fluorophore) and a non-detectable quencher moiety that quenches the fluorescence of the detectable label. In the presence of a target nucleic acid, the detectable label and the quencher separate which leads to an increase in signal emitted from the detectable label. Typically, a primer used in the amplification reaction includes a probe element at the 5′ end along with a “PCR blocker” element (e.g., a hexaethylene glycol (HEG) monomer (Whitcombe, et al. Nat. Biotech. 17: 804-807 (1999)) at the start of the hairpin loop. The probe typically includes a self-complementary stem sequence with a detectable label at one end and a quencher at the other. In the initial amplification cycles (e.g., PCR), the primer hybridizes to the target and extension occurs due to the action of polymerase. The Scorpions™ system may be used to examine and identify point mutations using multiple probes that may be differently tagged to distinguish between the probes. Using PCR as an example, after one extension cycle is complete, the newly synthesized target region will be attached to the same strand as the probe. Following the second cycle of denaturation and annealing, the probe and the target hybridize. The hairpin sequence then hybridizes to a part of the newly produced PCR product. This results in the separation of the detectable label from the quencher and causes emission of the signal. Other uses for such labeled probes are known in the art and would be suitable for use in the methods described herein.

In some embodiments, the methods are performed before or in conjunction with a sequencing reaction. The term “sequencing” is used in a broad sense herein and refers to any technique known in the art that allows the order of at least some consecutive nucleotides in at least part of a polynucleotide, for example but not limited to a target nucleic acid or an amplicon, to be identified. Some non-limiting examples of sequencing techniques include Sanger's dideoxy terminator method and the chemical cleavage method of Maxam and Gilbert, including variations of those methods; sequencing by hybridization; sequencing by synthesis; and restriction mapping. Some sequencing methods comprise electrophoresis, including capillary electrophoresis and gel electrophoresis; sequencing by hybridization including microarray hybridization; mass spectrometry; single molecule detection; and ion/proton detection. In some embodiments, sequencing comprises direct sequencing, duplex sequencing, cycle sequencing, single base extension sequencing (SBE), solid-phase sequencing, or combinations thereof. In some embodiments, sequencing comprises detecting the sequencing product using an instrument, for example but not limited to an ABI Prism® 377 DNA Sequencer, an ABI Prism® 310, 3100, 3100-Avant, 3730 or 3730×1 Genetic Analyzer, an ABI Prism® 3700 DNA Analyzer, an Ion PGM™ sequencer, or an Ion Proton™ sequencer (all available from Life Technologies Corp., Carlsbad, Calif.), or a mass spectrometer. In some embodiments, sequencing comprises incorporating a dNTP, including a dATP, a dCTP, a dGTP, a dTTP, a dUTP, a dITP, or combinations thereof, and including dideoxyribonucleotide analogs of dNTPs, into an amplification product.

The term “DNA polymerase” is used herein in a broad sense and refers to any polypeptide that can catalyze the 5′-to-3′ extension of a hybridized primer by the addition of deoxyribonucleotides and/or certain nucleotide analogs in a template-dependent manner. For example, but not limited to, the sequential addition of deoxyribonucleotides to the 3′-end of a primer that is annealed to a nucleic acid template during a primer extension reaction. Non-limiting examples of DNA polymerases include RNA-dependent DNA polymerases, including without limitation, reverse transcriptases, and DNA-dependent DNA polymerases. It is to be appreciated that certain DNA polymerases (for example, but not limited to certain eubacterial Type A DNA polymerases and Taq DNA polymerase) may further comprise a structure-specific nuclease activity and that when an amplification reaction comprises an invasive cleavage reaction.

In certain embodiments, a panel includes one or more of the following sequences: a sequence specific to Colton:

In some embodiments, the markers include one or more markers corresponding to alleles that determine the phenotypes of the ABO blood group system. The locus encodes glycosyltransferases that determine the carbohydrate antigens on band 3 protein or glycosphingolipid. In some embodiments, the markers correspond to ABO alleles A, B or O (negative phenotype). In some embodiments, the markers correspond to other mutations in the ABO coding or regulatory sequences.

In some embodiments, the markers include one or more markers corresponding to alleles encoding phenotypes of the Colton blood group system or Co antigen (also known as aquaporin-1), a membrane marker found on RBCs and kidney tubules. In some embodiments, the markers correspond to Colton alleles Co(a), Co(b) or Co-negative. In some embodiments, the markers correspond to other mutations in the Colton coding or regulatory sequences.

In some embodiments, the markers include one or more markers corresponding to alleles encoding phenotypes of the Cromer blood group system or CROM antigen (also known as decay-accelerating factor or DAF), a membrane marker that regulate complement activity found on RBCs. In some embodiments, the markers correspond to Cromer antigen Cr^{a}, SERF, ZENA or Inab (a Cromer negative phenotype). In some embodiments, the markers correspond to other mutations in the Cromer coding or regulatory sequences.

In some embodiments, the markers include one or more markers corresponding to alleles encoding phenotypes of the Diego (or Wright) blood group system (also known as band 3 protein, SLC4A1), a chloride/carbonate channel found on RBCs. In some embodiments, the markers correspond to Diego alleles Diego^{a} (Di^{a-} and Di^{a+}), Diego^{b} (Di^{b-} and Di^{b+}), Wr^{a} and Wr^{b}. In some embodiments, the markers correspond to other mutations in the Diego coding or regulatory sequences.

In some embodiments, the markers include one or more markers corresponding to alleles encoding phenotypes of the Dombrock or Do blood group system (also known as ART4 or CD297), a glycoprotein member of the ADP-ribosyltransferase gene family found on RBCs. In some embodiments, the markers correspond to Dombrock alleles Do^{a} and Do^{b}, Gy^{a}, Hy or Jo^{a}. In some embodiments, the markers correspond to other mutations in the Dombrock coding or regulatory sequences.

In some embodiments, the markers include one or more markers corresponding to alleles encoding phenotypes of the Duffy blood group system (also known as Fy glycoprotein or CD234), a surface marker found on RBCs. In some embodiments, the markers correspond to Duffy alleles FY*A, FY*B, FY*X and FY*Fy that encode for Fy-a antigen (Gly42), Fy-b antigen (Asp42), Fy-x phenotype (Cys89 and Thr100; or Ser49) and Fy-y antigens, respectively. In some embodiments, the markers correspond to a negative phenotype Fy-o, such as the Fy GATA promoter silencing mutation. In some embodiments, the markers correspond to other mutations in the Duffy coding or regulatory sequences.

In some embodiments, the markers include one or more markers corresponding to alleles encoding hemoglobin S or HbS (also known as sickle-cell hemoglobin).

In some embodiments, the markers include one or more markers corresponding to alleles encoding phenotypes of the Kell or K or Kp blood group system, a transmembrane zinc-dependent endopeptidase on RBCs. In some embodiments, the markers correspond to Kell antigens K1 (Kell), K2 (k or Cellano), Kpa, Kpb, Jsa, Jsb, and K null. In some embodiments, the markers correspond to other mutations in the Kell coding or regulatory sequences.

In some embodiments, the markers include one or more markers corresponding to alleles encoding phenotypes of the Knops or KN blood group system, a complement receptor 1 glycoprotein (CR1, CD35). In some embodiments, the markers correspond to other mutations in the Knops coding or regulatory sequences.

In some embodiments, the markers include one or more markers corresponding to alleles encoding phenotypes of the Landsteiner-Wiener or LW blood group system, a membrane glycoprotein (ICAM-4 or CD242) on RBCs. In some embodiments, the markers correspond to LW and LW null. In some embodiments, the markers correspond to other mutations in the LW coding or regulatory sequences.

In some embodiments, the markers include one or more markers corresponding to alleles encoding phenotypes of the Lutheran or LU blood group system, an immunoglobulin family member on RBCs. In some embodiments, the markers correspond to Lua and Lub null. In some embodiments, the markers correspond to other mutations in the LU coding or regulatory sequences.

In some embodiments, the markers include one or more markers corresponding to alleles encoding phenotypes of the MNS blood group system, based on glycophorin A (GYPA) and glycophorin B (GYPB) on RBCs. In some embodiments, the markers correspond to MNS antigens M and N (based on GYPA), S and s (based on GYPB), U (high frequency antigen, S- and s-), GYPB silencing +5intron5, GYPB silencing nt230. In some embodiments, the markers correspond to other mutations in the MNS coding or regulatory sequences.

In some embodiments, the markers include one or more markers corresponding to alleles encoding phenotypes of the MNS blood group system, based on glycophorin A (GYPA) and glycophorin B (GYPB) on RBCs. In some embodiments, the markers correspond to MNS antigens M and N (based on GYPA), S and s (based on GYPB), U (high frequency antigen, S- and s-), GYPB silencing +5intron5, GYPB silencing nt230, GYPB silencing nt143. In some embodiments, the markers correspond to other mutations in the MNS coding or regulatory sequences.

In some embodiments, the markers include one or more markers corresponding to alleles encoding phenotypes of the Rhesus or Rh blood group system on RBCs. In some embodiments, the markers correspond to Rh antigens Rh(C), Rh(c), Rh(E), Rh(e), Rh(D), Rh(D deletion), Rh(D 37 bp duplication or RHD-psi), Rh(D hybrid), Rh(C*), Rh(D-CE (4-7)-D hybrid) or null. In some embodiments, the markers correspond to other mutations in the Rh coding or regulatory sequences.

In some embodiments, the markers include one or more markers corresponding to alleles encoding phenotypes of the Scianna or SC blood group system on RBCs. In some embodiments, the markers correspond to Scianna antigens Sc1 and Sc2 or null. In some embodiments, the markers correspond to other mutations in the Scianna coding or regulatory sequences.

In some embodiments, the markers include one or more markers corresponding to alleles encoding phenotypes of the Yt or Cartwright blood group system, an acetylcholinesterase on RBCs. In some embodiments, the markers correspond to Yt antigens Yt(a) and Yt(b) or null. In some embodiments, the markers correspond to other mutations in the Yt coding or regulatory sequences.

In certain embodiments, methods and kits of the present disclosure include a panel for simultaneously determining the genotype of a plurality of blood groups. Exemplary panels in accordance with the present invention are shown in Table 1 (non-Rh panel) and Table 2 (Rh-panel).

TABLE 1
Exemplary NON-Rh PANEL

| Blood Group | Variant | dbSNP ID |
|---|---|---|
| Colton | Co a/b | rs28362692 |
| Cromer | Cromer a | rs60822373 |
| Diego | Di a/b | rs2285644 |
| Dombrock | Do a/b | rs11276 |
| Dombrock | Hy | rs28362797 |
| Dombrock | Joa | rs28362798 |
| Duffy | Fy a/b | rs12075 |
| Duffy | Fy GATA Silencing | rs2814778 |
| Duffy | Fyx | rs34599082 |
| Kell | Js a/b | rs8176038 |
| Kell | K/k | rs8176058 |
| Kell | Kp a/b | rs8176059 |
| Kidd | Jk a/b | rs1058396 |
| Knops | Kn a/b | rs41274768 |
| Landsteiner-Wiener | LW a/b | rs77493670 |
| Lutheran | Lu a/b | rs28399653 |
| MNS S/s | GYPB-Int 5 | |
| MNS M/N | GYPA nt 59C > T | rs7682260 |
| MNS M/N | GYPA nt 71-72AC > CT | rs71590431 |
| MNS S/s | GYPB 143 T > C | rs7683365 |
| MNS S/s | GYPB nt 230 | |
| Scianna | Sc a/b | rs79492560 |
| Yt | Yt a/b | rs56025238 |

TABLE 2
Exemplary Rh PANEL

| Blood Group | Variant | dbSNP ID |
|---|---|---|
| Rh - RHCE | RHCE L245V | rs1053361 |
| Rh - RHCE | RHCE G336C | rs116261244 |
| Rh - RHCE C/c | RHCE P103S | rs676785 |
| Rh - RHCE E/e | RHCE A226P | rs609320 |
| Rh - RHCE | RHCE W16C | rs586178 |
| Rh - RHCE | RHCE Cx | |
| Rh - RHCE | RHCE M238V | rs1053360 |
| Rh - RHCE C | RHCE intron 2 indel (109 | |
| | bp insertion) | |
| Rh - RHD | RHD 37 bp dupl. | |
| Rh - RHD | RHD gene del | |
| Rh - RHD | RHD hybrid gene* | |

The present disclosure includes a genotyping reference for use with the genotyping assay. The reference can be a nucleic acid that includes one or more sequences corresponding to known blood group alleles. Such references are particularly useful as certain alleles in the test panel (such as some of those in Tables 1 and 2) are rare in the population. Thus, the failure to detect a positive signal for a given marker may not be determinative of the absence of that marker in the nucleic sample, as opposed to a failure of the one or more steps of the reaction or one or more components of the panel. Likewise, the apparent positive signal for a given marker may not be determinative of the presence of that marker in the nucleic sample, as opposed to a false positive. Accordingly, the reference nucleic acid provides a known positive and negative controls, particularly for rare alleles.

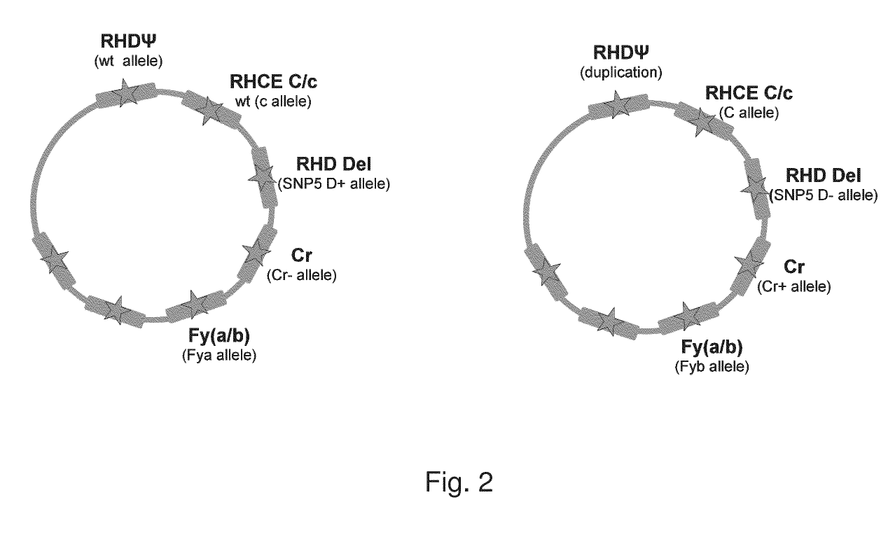

Exemplary nucleic acid controls are shown in FIG. 2. In some embodiments, reference comprises at least a pair of nucleic acids, in which each nucleic acid contains an allele that may be mutually exclusive to the allele in the other nucleic acid. In some embodiments, reference comprises at least a pair of nucleic acids, in which each nucleic acid contains one of two alleles of a single locus.

In some embodiments, the reference nucleic acid molecule can be a nucleic acid construct such as, for example, in the form of a plasmid, a viral vector, an artificial chromosome, or a nucleic acid fragment. The reference can, for example, be recombinantly generated or chemically synthesized. In some embodiments, the reference nucleic acid molecule includes amplicons synthesized from marker allele-containing portions of blood group loci and joined together in one nucleic acid molecule.

The reference molecule contains at least two marker alleles from blood group loci. In some embodiments, the reference molecule contains at least 3 marker alleles from blood group loci. In other embodiments, a reference nucleic acid molecule contains at least 4, at least 5, at least 6, at least 7, at least 8, at least 9, at least 10, at least 15 different marker alleles from blood group loci. In some embodiments, the reference nucleic acid molecule contains from 2 to 30 different marker alleles from blood group loci. In certain embodiments, the reference nucleic acid molecule contains from about 3 to about 25 different marker alleles from blood group loci. In other embodiments, the reference nucleic acid molecule contains from about 5 to about 20, about 6 to about 18, from about 8 to about 15, or from about 10 to about 13 different marker alleles from blood group loci. In some embodiments, the reference nucleic acid molecule contains about 3, about 5, about 7, about 9, about 12, about 14, about 16, about 18, or about 22 different marker alleles from blood group loci.

In some embodiments, the genotyping reference is at least a pair of nucleic acid molecules, in which each nucleic acid of the pair contains a different allele from a particular blood group locus. For example, in a given pair of reference nucleic acid molecules, one reference nucleic acid contains an RHCE C allele and the other reference nucleic acid contains the RHCE c allele. In another example, one nucleic acid molecule of a reference nucleic acid pair contains the Kell Js(a) and the Dombrook Do^{b} alleles and the other nucleic acid molecule of the pair contains the Kell Js(b) and the Dombrook Do^{a} alleles. In some embodiments, the reference can contain any combination of blood group marker alleles. In some embodiments, the genotyping reference contains the sequence of two or more alleles of the same blood group antigen. For example, without limitation, a reference nucleic acid molecule can contain a sequence for an MNS N allele and a sequence for an MNS S allele. In some embodiments, the genotyping reference is at least a set of three nucleic acid molecules wherein an allele from a particular blood group locus is present in only one of the nucleic acid molecules of the set.

The reference serves as positive and/or negative control for the blood group genotyping assays. In some embodiments, the genotyping reference provided herein can be used to calibrate blood group genotyping assays and/or systems for blood group genotyping. The combination of marker alleles in a reference nucleic acid molecule is selected based on the blood group alleles being determined in the genotyping assay. As such, when used in conjunction with a genotyping assays, the reference molecules used contain the same alleles to which the assays are directed. In some embodiments, the reference includes all of the alleles to which the particular assays are directed. In other embodiments, the reference includes a subset of the alleles to which the assays are directed.

In some embodiments, provided herein are nucleic acid genotyping assays where the alleles genotyped are sourced from a single nucleic acid molecule. In certain embodiments, provided herein are nucleic acid genotyping assays where at least three of the alleles genotyped are sourced from a single nucleic acid molecule. In other embodiments, provided herein are nucleic acid genotyping assays where at least five of the alleles genotyped are sourced from a single nucleic acid molecule. In other embodiments, provided herein are nucleic acid genotyping assays where at least seven of the alleles genotyped are sourced from a single nucleic acid molecule.

The nucleic acid polymerases that may be employed in the disclosed nucleic acid amplification reactions may be any that function to carry out the desired reaction including, for example, a prokaryotic, fungal, viral, bacteriophage, plant, and/or eukaryotic nucleic acid polymerase. As used herein, the term “DNA polymerase” refers to an enzyme that synthesizes a DNA strand de novo using a nucleic acid strand as a template. DNA polymerase uses an existing DNA or RNA as the template for DNA synthesis and catalyzes the polymerization of deoxyribonucleotides alongside the template strand, which it reads. The newly synthesized DNA strand is complementary to the template strand. DNA polymerase can add free nucleotides only to the 3′-hydroxyl end of the newly forming strand. It synthesizes oligonucleotides via transfer of a nucleoside monophosphate from a deoxyribonucleoside triphosphate (dNTP) to the 3′-hydroxyl group of a growing oligonucleotide chain. This results in elongation of the new strand in a 5′-to-3′ direction. Since DNA polymerase can only add a nucleotide onto a pre-existing 3′-OH group, to begin a DNA synthesis reaction, the DNA polymerase needs a primer to which it can add the first nucleotide. Suitable primers may comprise oligonucleotides of RNA or DNA, or chimeras thereof (e.g., RNA/DNA chimerical primers). The DNA polymerases may be a naturally occurring DNA polymerases or a variant of natural enzyme having the above-mentioned activity. For example, it may include a DNA polymerase having a strand displacement activity, a DNA polymerase lacking 5′-to-3′ exonuclease activity, a DNA polymerase having a reverse transcriptase activity, or a DNA polymerase having an endonuclease activity.

Suitable nucleic acid polymerases may also comprise holoenzymes, functional portions of the holoenzymes, chimeric polymerase, or any modified polymerase that can effectuate the synthesis of a nucleic acid molecule. Within this disclosure, a DNA polymerase may also include a polymerase, terminal transferase, reverse transcriptase, telomerase, and/or polynucleotide phosphorylase. Non-limiting examples of polymerases may include, for example, T7 DNA polymerase, eukaryotic mitochondrial DNA Polymerase γ, prokaryotic DNA polymerase I, II, III, IV, and/or V; eukaryotic polymerase α, β, γ, δ, ε, η, ζ, τ, and/or κ; E. coli DNA polymerase I; E. coli DNA polymerase III alpha and/or epsilon subunits; E. coli polymerase IV, E. coli polymerase V; T. aquaticus DNA polymerase I; B. stearothermophilus DNA polymerase I; Euryarchaeota polymerases; terminal deoxynucleotidyl transferase (TdT); S. cerevisiae polymerase 4; translesion synthesis polymerases; reverse transcriptase; and/or telomerase. Non-limiting examples of suitable thermostable DNA polymerases that may be used include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVENT™ DNA polymerase, Pyrococcus woosii (Pwo) DNA polymerase, Bacillus sterothermophilus (Bst) DNA polymerase, Bacillus caldophilus (Bca) DNA polymerase, Sulfobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), and mutants, and variants and derivatives thereof. RNA polymerases such as T3, T5 and SP6 and mutants, variants and derivatives thereof may also be used in accordance with the present teachings. Generally, any type I DNA polymerase may be used in accordance with the invention although other DNA polymerases may be used including, but not limited to, type III or family A, B, C etc. DNA polymerases. In addition, any genetically engineered DNA polymerases, any having reduced or insignificant 3′-to-5′ exonuclease activity (e.g., SuperScript™ DNA polymerase), and/or genetically engineered DNA polymerases (e.g., those having the active site mutation F667Y or the equivalent of F667Y (e.g., in Tth), AmpliTaq® FS, ThermoSequenase™), AmpliTaq® Gold, Platinum® Taq DNA Polymerase, Therminator I, Therminator II, Therminator III, Therminator Gamma (all available from New England Biolabs, Beverly, Mass.), and/or any derivatives and fragments thereof, may be used in accordance with the present teachings. Other nucleic acid polymerases may also be suitable as would be understood by one of skill in the art.

Polymerases used in accordance with the present teachings may be any enzyme that can synthesize a nucleic acid molecule from a nucleic acid template, typically in the 5′ to 3′ direction. The nucleic acid polymerases used in the methods disclosed herein may be mesophilic or thermophilic. Exemplary mesophilic DNA polymerases include T7 DNA polymerase, T5 DNA polymerase, Klenow fragment DNA polymerase, DNA polymerase III and the like. Exemplary thermostable DNA polymerases that may be used in the methods of the present teachings include Taq, Tne, Tma, Pfu, Tfl, Tth, Stoffel fragment, VENT™ and DEEPVENT™ DNA polymerases, and mutants, variants and derivatives thereof (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nucl. Acids Res. 22(15):3259-3260 (1994)). Examples of DNA polymerases substantially lacking in 3′ exonuclease activity include, but are not limited to, Taq, Tne(exo-), Tma(exo-), Pfu (exo-), Pwo(exo-) and Tth DNA polymerases, and mutants, variants and derivatives thereof.

DNA polymerases for use in the methods disclosed herein may be obtained commercially, for example, from Life Technologies Corp. (Carlsbad, Calif.), Pharmacia (Piscataway, N.J.), Sigma (St. Louis, Mo.) and Boehringer Mannheim. Exemplary commercially available DNA polymerases for use in the present invention include, but are not limited to, Tsp DNA polymerase from Life Technologies Corp.

Enzymes for use in the compositions, methods, compositions and kits provided herein may also include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, et al., Science 239:487-491 (1988); U.S. Pat. Nos. 4,889,818 and 4,965,188), Tne DNA polymerase (WO 96/10640), Tma DNA polymerase (U.S. Pat. No. 5,374,553) and mutants, fragments, variants or derivatives thereof (see, e.g., commonly owned, co-pending U.S. patent application Ser. Nos. 08/706,702 and 08/706,706, both filed Sep. 9, 1996, which are incorporated by reference herein in their entireties). As will be understood by one of ordinary skill in the art, modified reverse transcriptases and DNA polymerase having reverse transcriptase activity may be obtained by recombinant or genetic engineering techniques that are well-known in the art. Mutant reverse transcriptases or polymerases may, for example, be obtained by mutating the gene or genes encoding the reverse transcriptase or polymerase of interest by site-directed or random mutagenesis. Such mutations may include point mutations, deletion mutations and insertional mutations. In some embodiments, one or more point mutations (e.g., substitution of one or more amino acids with one or more different amino acids) are used to construct mutant reverse transcriptases or polymerases for use in the invention. Fragments of reverse transcriptases or polymerases may also be obtained by deletion mutation by recombinant techniques that are well-known in the art, or by enzymatic digestion of the reverse transcriptase(s) or polymerase(s) of interest using any of a number of well-known proteolytic enzymes.

In some embodiments, enzymes for use in the methods provided herein include those that are reduced or substantially reduced in RNase H activity. Such enzymes that are reduced or substantially reduced in RNase H activity may be obtained by mutating the RNase H domain within the reverse transcriptase of interest, for example, by one or more point mutations, one or more deletion mutations, or one or more insertion mutations as described above. An enzyme “substantially reduced in RNase H activity” refers to an enzyme that has less than about 30%, less than about 25%, less than about 20%, less than about 15%, less than about 10%, less than about 7.5%, or less than about 5%, or less than about 5% or less than about 2%, of the RNase H activity of the corresponding wild type or RNase H⁺ enzyme such as wild type Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, et al., Nucl. Acids Res. 16:265 (1988), in Gerard, et al., FOCUS 14(5):91 (1992), and in U.S. Pat. No. 5,668,005, the disclosures of all of which are fully incorporated herein by reference.

Polypeptides having reverse transcriptase activity for use in the methods provided herein may be obtained commercially, for example, from Life Technologies Corp. (Carlsbad, Calif.), Pharmacia (Piscataway, N.J.), Sigma (Saint Louis, Mo.) or Boehringer Mannheim Biochemicals (Indianapolis, Ind.). Alternatively, polypeptides having reverse transcriptase activity may be isolated from their natural viral or bacterial sources according to standard procedures for isolating and purifying natural proteins that are well-known to one of ordinary skill in the art (see, e.g., Houts, et al., J. Virol. 29:517 (1979)). In addition, the polypeptides having reverse transcriptase activity may be prepared by recombinant DNA techniques that are familiar to one of ordinary skill in the art (see, e.g., Kotewicz, et al., Nucl. Acids Res. 16:265 (1988); Soltis and Skalka, Proc. Natl. Acad. Sci. USA 85:3372-3376 (1988)).

Exemplary polypeptides having reverse transcriptase activity for use in the methods provided herein include M-MLV reverse transcriptase, RSV reverse transcriptase, AMV reverse transcriptase, Rous Associated Virus (RAV) reverse transcriptase, Myeloblastosis Associated Virus (MAV) reverse transcriptase and Human Immunodeficiency Virus (HIV) reverse transcriptase, and others described in WO 98/47921 and derivatives, variants, fragments or mutants thereof, and combinations thereof. In a further embodiment, the reverse transcriptases are reduced or substantially reduced in RNase H activity, and may be selected from the group consisting of M-MLV H− reverse transcriptase, RSV H− reverse transcriptase, AMV H− reverse transcriptase, RAV H− reverse transcriptase, MAV H− reverse transcriptase and HIV H− reverse transcriptase, and derivatives, variants, fragments or mutants thereof, and combinations thereof. Reverse transcriptases of particular interest include AMV RT and M-MLV RT, and optionally AMV RT and M-MLV RT having reduced or substantially reduced RNase H activity (e.g., AMV RT alpha H−/BH+ and M-MLV RT H−). Reverse transcriptases for use in the invention include SuperScript™ SuperScript™II, ThermoScript™ and ThermoScript™ II available from Life Technologies Corp. See generally, WO 98/47921, U.S. Pat. Nos. 5,244,797 and 5,668,005, the entire contents of each of which are herein incorporated by reference.

In another aspect, the present disclosure provides reaction mixtures for polymerizing and/or amplifying a nucleic acid sequence of interest (e.g., a target sequence). In some embodiments, the reaction mixture may further comprise a detectable label. The methods may also include one or more steps for detecting the detectable label to quantitate the amplified nucleic acid. As used herein, the term “detectable label” refers to any of a variety of signaling molecules indicative of amplification. For example, SYBR® Green and other DNA-binding dyes are detectable labels. Such detectable labels may comprise or may be, for example, nucleic acid intercalating agents or non-intercalating agents. As used herein, an intercalating agent is an agent or moiety capable of non-covalent insertion between stacked base pairs of a double-stranded nucleic acid molecule. A non-intercalating agent is one that does not insert into the double-stranded nucleic acid molecule. The nucleic acid binding agent may produce a detectable signal directly or indirectly. The signal may be detectable directly using, for example, fluorescence and/or absorbance, or indirectly using, for example, any moiety or ligand that is detectably affected by proximity to double-stranded nucleic acid is suitable such as a substituted label moiety or binding ligand attached to the nucleic acid binding agent. It is typically necessary for the nucleic acid binding agent to produce a detectable signal when bound to a double-stranded nucleic acid that is distinguishable from the signal produced when that same agent is in solution or bound to a single-stranded nucleic acid. For example, intercalating agents such as ethidium bromide fluoresce more intensely when intercalated into double-stranded DNA than when bound to single-stranded DNA, RNA, or in solution (see, e.g., U.S. Pat. Nos. 5,994,056; 6,171,785; and/or 6,814,934). Similarly, actinomycin D fluoresces in the red portion of the UV/VIS spectrum when bound to single-stranded nucleic acids, and fluoresces in the green portion of the UV/VIS spectrum when bound to double-stranded nucleic acids. And in another example, the photoreactive psoralen 4-aminomethyl-4-5′,8-trimethylpsoralen (AMT) has been reported to exhibit decreased absorption at long wavelengths and fluorescence upon intercalation into double-stranded DNA (Johnson et al. Photochem. & Photobiol., 33:785-791 (1981). For example, U.S. Pat. No. 4,257,774 describes the direct binding of fluorescent intercalators to DNA (e.g., ethidium salts, daunomycin, mepacrine and acridine orange, 4′,6-diamidino-α-phenylindole). Non-intercalating agents (e.g., minor groove binders as described herein such as Hoechst 33258, distamycin, netropsin) may also be suitable for use. For example, Hoechst 33258 (Searle, et al. Nucl. Acids Res. 18(13):3753-3762 (1990)) exhibits altered fluorescence with an increasing amount of target. Minor groove binders are described in more detail elsewhere herein.

Other DNA binding dyes are available to one of skill in the art and may be used alone or in combination with other agents and/or components of an assay system. Exemplary DNA binding dyes may include, for example, acridines (e.g., acridine orange, acriflavine), actinomycin D (Jain, et al. J. Mol. Biol. 68:21 (1972)), anthramycin, BOBO™-1, BOBO™-3, BO-PRO™-1, cbromomycin, DAPI (Kapuseinski, et al. Nucl. Acids Res. 6(112): 3519 (1979)), daunomycin, distamycin (e.g., distamycin D), dyes described in U.S. Pat. No. 7,387,887, ellipticine, ethidium salts (e.g., ethidium bromide), fluorcoumanin, fluorescent intercalators as described in U.S. Pat. No. 4,257,774, GelStar® (Cambrex Bio Science Rockland Inc., Rockland, Me.), Hoechst 33258 (Searle and Embrey, Nucl. Acids Res. 18:3753-3762 (1990)), Hoechst 33342, homidium, JO-PRO™-1, LIZ dyes, LO-PRO™-1, mepacrine, mithramycin, NED dyes, netropsin, 4′,6-diamidino-α-phenylindole, proflavine, POPO™-1, POPO™-3, PO-PRO™-1, propidium iodide, ruthenium polypyridyls, S5, SYBR® Gold, SYBR® Green I (U.S. Pat. Nos. 5,436,134 and 5,658,751), SYBR® Green II, SYTOX® blue, SYTOX® green, SYTO® 43, SYTO® 44, SYTO® 45, SYTOX® Blue, TO-PRO®-1, SYTO® 11, SYTO® 13, SYTO® 15, SYTO® 16, SYTO® 20, SYTO® 23, thiazole orange (Aldrich Chemical Co., Milwaukee, Wis.), TOTO™-3, YO-PRO®-1, and YOYO®-3 (Molecular Probes, Inc., Eugene, Oreg.), among others. SYBR® Green I (see, e.g., U.S. Pat. Nos. 5,436,134; 5,658,751; and/or 6,569,927), for example, has been used to monitor a PCR reactions. Other DNA binding dyes may also be suitable as would be understood by one of skill in the art.

For use as described herein, one or more detectable labels and/or quenching agents may be attached to one or more primers and/or probes (e.g., detectable label). The detectable label may emit a signal when free or when bound to one of the target nucleic acids. The detectable label may also emit a signal when in proximity to another detectable label. Detectable labels may also be used with quencher molecules such that the signal is only detectable when not in sufficiently close proximity to the quencher molecule. For instance, in some embodiments, the assay system may cause the detectable label to be liberated from the quenching molecule. Any of several detectable labels may be used to label the primers and probes used in the methods described herein. As mentioned above, in some embodiments the detectable label may be attached to a probe, which may be incorporated into a primer, or may otherwise bind to amplified target nucleic acid (e.g., a detectable nucleic acid binding agent such as an intercalating or non-intercalating dye). When using more than one detectable label, each should differ in their spectral properties such that the labels may be distinguished from each other, or such that together the detectable labels emit a signal that is not emitted by either detectable label alone. Exemplary detectable labels include, for instance, a fluorescent dye or fluorphore (e.g., a chemical group that can be excited by light to emit fluorescence or phosphorescence), “acceptor dyes” capable of quenching a fluorescent signal from a fluorescent donor dye, and the like. Suitable detectable labels may include, for example, fluorosceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); FITC; 6-carboxy-1,4-dichloro-2′,7′-dichlorofluorescein (TET); 6-carboxy-1,4-dichloro-2′,4′,5′,7′-tetra-chlorofluorescein (HEX); 6-carboxy-4′,5′-dichloro-2′,7′-dimethoxyfluorescein (JOE); Alexa fluor® fluorophores (e.g., 350, 405, 430, 488, 500, 514, 532, 546, 555, 568, 594, 610, 633, 635, 647, 660, 680, 700, 750); BODIPY® fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), coumarins (e.g., 7-amino-4-methylcoumarin, AMC, AMCA, AMCA-S, AMCA-X, ABQ, CPM methylcoumarin, coumarin phalloidin, hydroxycoumarin, CMFDA, methoxycoumarin), calcein, calcein AM, calcein blue, calcium dyes (e.g., calcium crimson, calcium green, calcium orange, calcofluor white), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/tetramethylrhodamine, IAEDANS/fluorescein, EDANS/dabcyl, fluorescein/fluorescein, BODIPY® FL/BODIPY® FL, Fluorescein/QSY7 and QSY9), LysoTracker® and LysoSensor™ (e.g., LysoTracker® Blue DND-22, LysoTracker® Blue-White DPX, LysoTracker® Yellow HCK-123, LysoTracker® Green DND-26, LysoTracker® Red DND-99, LysoSensor™ Blue DND-167, LysoSensor™ Green DND-189, LysoSensor™ Green DND-153, LysoSensor™ Yellow/Blue DND-160, LysoSensor™ Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethylrhodamine), Tetramethylrhodamine (TRITC), WT), Texas Red, Texas Red-X, VIC and other labels described in, e.g., U.S. Patent Application Publication No. 2009/0197254 (incorporated herein by reference in its entirety), among others as would be known to those of skill in the art. Other detectable labels may also be used (see, e.g., U.S. Patent Application Publication No. 2009/0197254 (incorporated herein by reference in its entirety)), as would be known to those of skill in the art. Any of these systems and detectable labels, as well as many others, may be used to detect amplified target nucleic acids.

Some detectable labels may be sequence-based (also referred to herein as “locus-specific detectable label”), for example 5′-nuclease probes. Such probes may comprise one or more detectable labels. Various detectable labels are known in the art, for example (TaqMan® probes described herein (See also U.S. Pat. No. 5,538,848 (incorporated herein by reference in its entirety)) various stem-loop molecular beacons (See, e.g., U.S. Pat. Nos. 6,103,476 and 5,925,517 and Tyagi and Kramer, Nature Biotechnology 14:303-308 (1996)), stemless or linear beacons (See, e.g., PCT Publication No. WO 99/21881; U.S. Pat. No. 6,485,901), PNA Molecular Beacons™ (See, e.g., U.S. Pat. Nos. 6,355,421 and 6,593,091), linear PNA beacons (See, e.g., Kubista et al., SPIE 4264:53-58 (2001)), non-FRET probes (See, e.g., U.S. Pat. No. 6,150,097), Sunrise®/Amplifluor® probes (U.S. Pat. No. 6,548,250), stem-loop and duplex Scorpions™ probes (Solinas et al., Nucleic Acids Research 29:E96 (2001) and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes (Svanvik, et al. Anal Biochem 281:26-35 (2001)), self-assembled nanoparticle probes, ferrocene-modified probes described, for example, in U.S. Pat. No. 6,485,901; Mhlanga et al., Methods 25:463-471 (2001); Whitcombe et al., Nature Biotechnology. 17:804-807 (1999); Isacsson et al., Molecular Cell Probes. 14:321-328 (2000); Svanvik et al., Anal Biochem. 281:26-35 (2000); Wolffs et al., Biotechniques 766:769-771 (2001); Tsourkas et al., Nucleic Acids Research. 30:4208-4215 (2002); Riccelli et al., Nucleic Acids Research 30:4088-4093 (2002); Zhang et al., Acta Biochimica et Biophysica Sinica (Shanghai). 34:329-332 (2002); Maxwell et al., J. Am. Chem. Soc. 124:9606-9612 (2002); Broude et al., Trends Biotechnol. 20:249-56 (2002); Huang et al., Chem Res. Toxicol. 15:118-126 (2002); and Yu et al., J. Am. Chem. Soc. 14:11155-11161 (2001); QuantiProbes® (www.qiagen.com), HyBeacons® (French, et al. Mol. Cell. Probes 15:363-374 (2001)), displacement probes (Li, et al. Nucl. Acids Res. 30:e5 (2002)), HybProbes (Cardullo, et al. Proc. Natl. Acad. Sci. USA 85:8790-8794 (1988)), MGB Alert (www.nanogen.com), Q-PNA (Fiandaca, et al. Genome Res. 11:609-611 (2001)), Plexor® (www.Promega.com), LUX™ primers (Nazarenko, et al. Nucleic Acids Res. 30:e37 (2002)), DzyNA primers (Todd, et al. Clin. Chem. 46:625-630 (2000)). Detectable labels may also comprise non-detectable quencher moieties that quench the fluorescence of the detectable label, inlcuding, for example, black hole quenchers (Biosearch), Iowa Black® quenchers (IDT), QSY quencher (Molecular Probes), and Dabsyl and Dabcyl sulfonate/carboxylate Quenchers (Epoch). Detectable labels may also comprise two probes, wherein for example a fluorophore is on one probe, and a quencher is on the other, wherein hybridization of the two probes together on a target quenches the signal, or wherein hybridization on a target alters the signal signature via a change in fluorescence. Exemplary systems may also include FRET, salicylate/DTPA ligand systems (see, e.g., Oser et al. Angew. Chem. Int. Engl. 29(10):1167 (1990)), displacement hybridization, homologous probes, and/or assays described in European Patent No. EP 070685 and/or U.S. Pat. No. 6,238,927. Detectable labels can also comprise sulfonate derivatives of fluorescein dyes with SO₃ instead of the carboxylate group, phosphoramidite forms of fluorescein, phosphoramidite forms of Cy5 (available for example from Amersham). All references cited above are hereby incorporated herein by reference in their entirety.

The compositions and methods described herein may be useful for detecting and/or quantifying a variety of target nucleic acids from a test sample. A target nucleic acid is any nucleic acid for which an assay system is designed to identify or detect as present (or not), and/or quantify in a test sample. Such nucleic acids may include, for example, those of infectious agents (e.g., virus, bacteria, parasite, and the like), a disease process such as cancer, diabetes, or the like, or to measure an immune response. Exemplary “test samples” include various types of samples, such as biological samples. Exemplary biological samples include, for instance, a bodily fluid (e.g., blood, saliva, spinal fluid), a tissue sample, a food (e.g., meat) or beverage (e.g., milk) product, or the like. Expressed nucleic acids may include, for example, genes for which expression (or lack thereof) is associated with medical conditions such as infectious disease (e.g., bacterial, viral, fungal, protozoal infections) or cancer. The methods described herein may also be used to detect contaminants (e.g., bacteria, virus, fungus, and/or protozoan) in pharmaceutical, food, or beverage products. The methods described herein may be also be used to detect rare alleles in the presence of wild type alleles (e.g., one mutant allele in the presence of 10⁶-10⁹ wild type alleles). The methods are useful to, for example, detect minimal residual disease (e.g., rare remaining cancer cells during remission, especially mutations in the p53 gene or other tumor suppressor genes previously identified within the tumors), and/or measure mutation load (e.g., the frequency of specific somatic mutations present in normal tissues, such as blood or urine).

The detection of the signal may be using any reagents or instruments that detect a change in fluorescence from a fluorophore. For example, detection may be performed using any spectrophotometric thermal cycler. Examples of spectrophotometric thermal cyclers include, but are not limited to, Applied Biosystems (AB) PRISM® 7000, AB 7300 real-time PCR system, AB 7500 real-time PCR system, AB PRISM® 7900HT, Bio-Rad ICycler IQ™ Cepheid SmartCycler® II, Corbett Research Rotor-Gene 3000, Idaho Technologies R.A.P.I.D.™, MJ Research Chromo 4™, Roche Applied Science LightCycler®, Roche Applied Science LightCycler® 2.0, Stratagene Mx3000P™, and Stratagene Mx4000™. It should be noted that new instruments are being developed at a rapid rate and any like instruments may be used for the methods.

Kits for performing the methods described herein are also provided. As used herein, the term “kit” refers to a packaged set of related components, typically one or more compounds or compositions. The kit may comprise a pair of oligonucleotides for polymerizing and/or amplifying at least one target nucleic acid from a sample, one or more detergents, a nucleic acid polymerase, and/or corresponding one or more probes labeled with a detectable label. The kit may also include samples containing pre-defined target nucleic acids to be used in control reactions. The kit may also optionally include stock solutions, buffers, enzymes, detectable labels or reagents required for detection, tubes, membranes, and the like that may be used to complete the amplification reaction. In some embodiments, multiple primer sets are included. In one embodiment, the kit may include one or more of, for example, a buffer (e.g., Tris), one or more salts (e.g., KCl), glycerol, dNTPs (dA, dT, dG, dC, dU), recombinant BSA (bovine serum albumin), a dye (e.g., ROX passive reference dye), one or more detergents, polyethylene glycol (PEG), polyvinyl pyrrolidone (PVP), and/or gelatin (e.g., fish or bovine source). Other embodiments of particular systems and kits are also contemplated which would be understood by one of skill in the art.

The methods and compositions may be used for detection and quantification of nucleic acids in a sample. The sample may include one or more templates and/or one or more target nucleic acids. The sample may be purified or unpurified. The sample may be a biological sample, such as blood, saliva, tears, tissue, urine, stool, etc., that has been treated to use in the methods provided herein. Alternatively, if the biological sample does not interfere with the methods provided herein, it may be used untreated (or unpurified).

While the present teachings have been described in terms of these exemplary embodiments, the skilled artisan will readily understand that numerous variations and modifications of these exemplary embodiments are possible without undue experimentation. All such variations and modifications are within the scope of the current teachings. Aspects of the present teachings may be further understood in light of the following examples, which should not be construed as limiting the scope of the teachings in any way.

### EXAMPLE

A panel of TaqMan® SNP Genotyping Assays were designed to detect the alleles of single nucleotide polymorphism (SNP) based blood group antigens. The panel of assays was designed to discriminate between alleles of 29 SNPs associated with 40 antigens in 14 blood groups (Co, Cr, Di, Do, Fy, Kel, Jk, Kn, LW, Lu, MNS, Sc, Yt, and Rh). TaqMan® OpenArray® plates (Applied Biosystems) for the QuantStudio™ 12K Flex Real-Time PCR System (Applied Biosystems) were pre-spotted with the panel of genotyping assays and stored at −20° C. until use in the assay. Each genotyping assay included a pair of amplification primers and two oligonucleotide TaqMan® probes, each probe with a different detectable label. The pair of amplification primers was specific for a portion of a blood group locus including at least one allele. Each probe was specific for one allele of the blood group locus and one probe labeled with VIC and the other probe labeled with FAM.

DNA was prepared from whole blood samples using a QIAcube® (Qiagen) or Universal BioRobot® (Qiagen) and the QIAamp® DNA Blood Kit per manufacturer's instructions, followed by quantification using a NanoDrop™ spectrophotometer (Thermo Scientific). DNA samples prepared using QIAcube® were diluted to a final concentration of 50 ng/mL. DNA samples prepared using Universal BioRobot® were diluted to 0.4-4 ng/mL and then followed by a pre-amplification reaction. The amount of DNA needed for an assay using an OpenArray® plate is 2 μL per sample at a concentration of 50 ng/μL, totaling 100 ng or 2 μL of diluted preamplification product.

The DNA samples and PCR master mix were loaded on the OpenArray® plates using an OpenArray Accufill System and run on the QuantStudio™ 12K Flex system in SNP Genotyping mode per manufacturer's instructions. The instrument has a built-in protocol for experiments to be run in SNP Genotyping mode. After the run, data files were uploaded to TaqMan® Genotyper Software and analyzed. TaqMan® Genotyper Software generated the genotypes associated with blood group antigens of the DNA samples. Genotype calls were exported using the advanced export option for downstream analysis with AlleleTyper™ Software.

AlleleTyper™ Software takes a user defined translation table as a template to convert blood group genotypes to blood group phenotypes. The translation table was uploaded to the software. The advanced export file from TaqMan® Genotyper Software serves as input data file for AlleleTyper™ Software. AlleleTyper™ Software generated the sample phenotype, showing the predicted presence (+) of some blood group antigens and absence (0) of other blood group antigens.

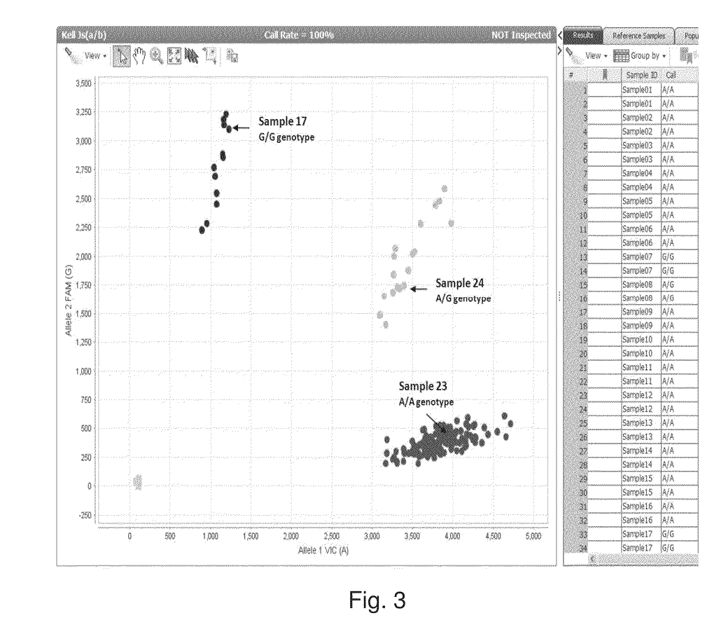

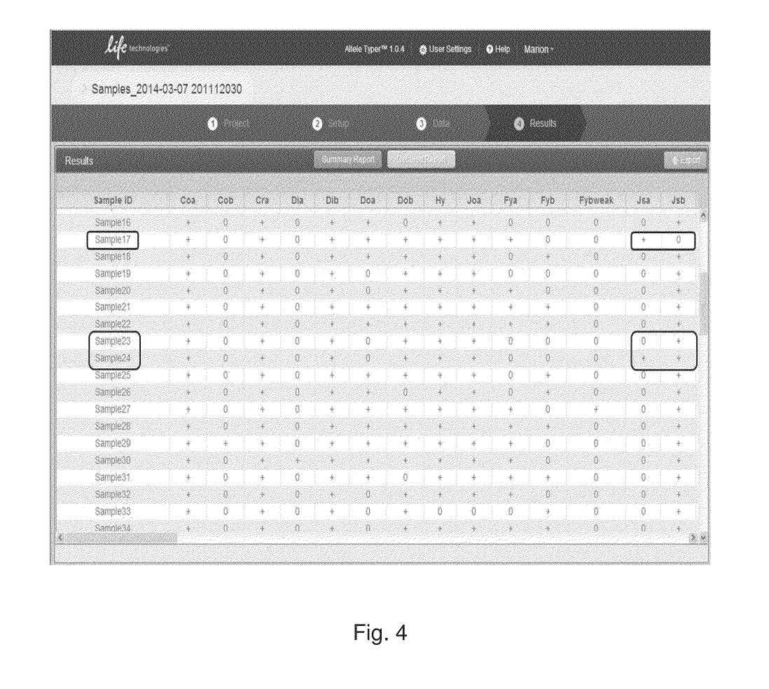

FIG. 3 and FIG. 4 show examples of genotype calling and phenotype calling of the Kell Js(a/b) blood group antigen. FIG. 3 shows representative allelic discrimination plots for the Kell Js(a/b) blood group antigens in which clusters are clearly defined for each genotype. For example, Sample 17 has a homozygous G/G genotype (FIG. 3) indicating the presence of the Jsa antigen and the absence of the Jsb antigen, and this genotype call was translated to a Js(a+/b−), displayed as Jsa+Jsb 0, phenotype by the AlleleTyper™ Software (FIG. 4). Sample 24 has a heterozygous A/G genotype translating to a Js(a+/b+) phenotype, and Sample 23 has a homozygous A/A genotype translating to a Js(a−/b+) phenotype. To assess concordance of the results with that from other genotyping methods like the BioArray™ HEA Beadchip™, sequencing or gel-based assays, over 12,000 known genotypes were typed with this panel. The overall concordance of this panel with other genotyping methods was 99.9%. Twenty-two assays showed 100% concordance with the other the other methods, and six assays showed concordance ranging from 99.14% to 99.80%. One assay showed a lower concordance of 90% (1 in 10 samples discordant) due to the low number of samples (10) available with known genotypes and this concordance result is statistically not significant.

What is claimed is:

1. A method for genotyping a nucleic acid sample, the method comprising:
performing a plurality of individual amplification reactions on the nucleic acid sample to form a plurality of individual amplification products,
wherein each amplification reaction comprises a pair of amplification primers specific for at least portions of a blood group locus sequence that flank at least one marker allele, wherein an individual amplification reaction is performed for a group of blood group loci consisting at least of: Colton, Cromer, Diego, Dombrock, Duffy, Kell, Kidd, Knops, Landsteiner-Wiener, Lutheran, MNS M/N, MNS S/s, Scianna, and Yt; and
determining the genotype of the blood group loci corresponding to the amplification products.

2. The method of claim 1, wherein the plurality of individual amplification reactions further comprises an individual amplification reaction for the ABO blood group.

3. The method of claim 1 or claim 2, wherein the plurality of individual amplification reactions further comprises an individual amplification reaction for at least one Rh blood group.

4. The method of any one of claims 1-3, wherein the performing amplification reactions is performed concurrently for each of the plurality of individual amplification reactions.

5. The method of any one of claims 1-4, wherein the determining the genotype is performed concurrently for each of the plurality of individual reaction products.

6. The method of any one of claims 1-5, wherein each amplification reaction further comprises at least one probe specific for a marker allele of the blood group locus amplified by the amplification primer pair.

7. The method of claim 6, wherein each amplification reaction comprises two probes, wherein each probe is specific for one marker allele of the blood group locus amplified by the amplification primer pair.

8. A high throughput method for genotyping a nucleic acid sample, the method comprising:
performing multiple parallel amplification reactions on a nucleic acid sample in individual reaction chambers to form amplification products in the individual reaction chambers,
wherein each amplification reaction comprises a pair of amplification primers specific for at least portions of a blood group locus sequence that flank at least one marker allele, wherein an amplification reaction is performed for each of the blood groups: Colton, Cromer, Diego, Dombrock, Duffy, Kell, Kidd, Knops, Landsteiner-Wiener, Lutheran, MNS M/N, MNS S/s, Scianna, and Yt; and
determining the genotype of the blood group loci corresponding to the amplification products.

9. The method of claim 8, wherein the multiple parallel amplification reactions further comprises an amplification reaction for the ABO blood group.

10. The method of claim 8 or claim 9, wherein the multiple parallel amplification reactions further comprises an amplification reaction for at least one Rh blood group.

11. The method of any one of claims 8-10, wherein each amplification reaction further comprises at least one probe specific for a marker allele of the blood group locus amplified by the amplification primer pair.

12. The method of claim 11, wherein each amplification reaction comprises two probes, wherein each probe is specific for one marker allele of the blood group locus amplified by the amplification primer pair and wherein each probe is labeled with a different detectable label.

13. The method of any one of claims 8-12, wherein the amplification reactions occur on a substrate comprising a plurality of through-hole reaction sites, wherein each site contains one of the amplification primer pairs.

14. A method for genotyping a nucleic acid sample, the method comprising:
performing an amplification reaction on the nucleic acid sample with a plurality of amplification primer pairs to form a plurality of amplification products,
wherein each amplification primer pair is specific for at least a portion of a sequence of a blood group locus, wherein the blood groups are selected from the group consisting of: ABO, Colton, Cromer, Diego, Dombrock, Duffy, hemoglobin S, Kell, Kidd, Knops, Landsteiner-Wiener, Lutheran, MNS M/N, MNS S/s, Rh, Scianna, and Yt; and
determining the genotype of the loci corresponding to the amplification products.

15. The method of claim 14, wherein the performing the amplification reaction step is performed concurrently for each of the plurality of amplification primer pairs.

16. The method of claim 14 or 15, wherein the determining the genotype step is performed concurrently for each of the amplification products.

17. The method of any one of claims 14-16, wherein the determining step comprises determining at least a portion of the nucleic acid sequence of at least one of the loci.

18. The method of any one of claims 14-17, wherein the determining step comprises determining at least a copy number of at least one of the loci.

19. The method of any one of claims 14-18, wherein one or more of the blood group loci to be amplified is the ABO blood group.

20. The method of claim 19, wherein the determining the genotype comprises determining the genotype of an allele corresponding to the A, B or O variants of the ABO blood group.

21. The method of any one of claims 14-20, wherein one or more of the blood group loci to be amplified is the Colton blood group.

22. The method of claim 21, wherein the determining the genotype comprises determining the genotype of an allele corresponding to the Co(a) or Co(b) variants of the Colton blood group.

23. The method of any one of claims 14-22, wherein one or more of the blood group loci to be amplified is the Cromer blood group.

24. The method of claim 23, wherein the determining the genotype comprises determining the genotype of an allele corresponding to the Co(a) or Co(b) variants of the Colton blood group.

25. The method of any one of claims 14-24, wherein one or more of the blood group loci to be amplified is the Cromer blood group.

26. The method of claim 25, wherein the determining the genotype comprises determining the genotype of an allele corresponding to the Cra (Cromer a), SERF, ZENA or Inab variants of the Cromer blood group.

27. The method of any one of claims 14-26, wherein one or more of the blood group loci to be amplified is the Diego blood group.

28. The method of claim 27, wherein the determining the genotype comprises determining the genotype of an allele corresponding to the Di(a), Di(b), Wr(a) or Wr(b) antigen of the Diego blood group.

29. The method of any one of claims 14-28, wherein one or more of the blood group loci to be amplified is the Dombrock blood group.

30. The method of claim 29, wherein the determining the genotype comprises determining the genotype of an allele corresponding to the Do^{a}, Do^{b}, Gy^{a}, Hy or Jo^{a} variants of the Dombrock blood group.

31. The method of any one of claims 14-30, wherein one or more of the blood group loci to be amplified is the Duffy blood group.

32. The method of claim 31, wherein the determining the genotype comprises determining the genotype of an allele corresponding to the Fy-a ,Fy-b, Fy-x, Fy-y, Fy(GATA silencing) or Fy-o variants of the Duffy blood group.

33. The method of any one of claims 14-32, wherein one or more of the blood group loci to be amplified is the hemoglobin S blood group.

34. The method of any one of claims 33, wherein the determining the genotype step comprises determining the genotype of an allele corresponding to the hemoglobin S (sickle cell) antigen.

35. The method of any one of claims 14-34, wherein one or more of the blood group loci to be amplified is the Kell blood group.

36. The method of claim 35, wherein the determining the genotype comprises determining the genotype of an allele corresponding to the K1 (K or Kell), K2 (k or Cellano), Kp(a), Kp(b), Js(a) or Js(b) variants of the Kell blood group.

37. The method of any one of claims 14-36, wherein one or more of the blood group loci to be amplified is the Kidd blood group.

38. The method of claim 37, wherein the determining the genotype comprises determining the genotype of an allele corresponding to the Jk(a) or Jk(b) variants of the Kidd blood group.

39. The method of any one of claims 14-38, wherein one or more of the blood group loci to be amplified is the Knops blood group.

40. The method of claim 39, wherein the determining the genotypecomprises determining the genotype of an allele corresponding to the Kn(a) or Kn(b) variants of the Knops blood group.

41. The method of any one of claims 14-40, wherein one or more of the blood group loci to be amplified is the Landsteiner-Wiener blood group.

42. The method of claim 41, wherein the determining the genotype comprises determining the genotype of an allele corresponding to the Lw(a) or Lw(b) variants of the Landsteiner-Wiener blood group.

43. The method of any one of claims 14-42, wherein one or more of the blood group loci to be amplified is the Lutheran blood group.

44. The method of claim 43, wherein the determining the genotype comprises determining the genotype of an allele corresponding to the Lu(a) or Lu(b) variants of the Lutheran blood group.

45. The method of any one of claims 14-44, wherein one or more of the blood group loci to be amplified is the MNS blood group.

46. The method of claim 45, wherein the determining the genotype comprises determining the genotype of an allele corresponding to the M and N (based on GYPA), S and s (based on GYPB), U (high frequency variants, S- and s-), GYPB silencing +5intron5, GYPB silencing nt230 variants of the MNS blood group.

47. The method of any one of claims 14-46, wherein one or more of the blood group loci to be amplified is the Rh blood group.

48. The method of claim 47, wherein the determining the genotype comprises determining the genotype of an allele corresponding to the RhCE(C), RhCE(c), RhCE(E), RhCE(e), RhCE L245V, RhCE G336C, RhCE W16C, RhCE M238V, RhCE(Cx), RHD (deletion), RhD (37 bp duplication or RHD-psi), Rh(D-CE (4-7)-D hybrid) or null variants of the Rh blood group.

49. The method of any one of claims 14-48, wherein one or more of the blood group loci to be amplified is the Scianna blood group.

50. The method of claim 49, wherein the determining the genotype comprises determining the genotype of anallele corresponding to the Sc(a) or Sc1 or Sc2 or Sc(b) variants of the Scianna blood group.

51. The method of any one of claims 14-50, wherein one or more of the blood group loci to be amplified is the Yt blood group.

52. The method of claim 51, wherein the determining the genotype comprises determining the genotype of anallele corresponding to the Yt(a) and Yt(b) variants of the Yt blood group.

53. The method of any one of claims 14-17, wherein blood group loci to be amplified include at least one locus of each of the Colton, Cromer, Diego, Dombrock, Duffy, Kell, Kidd, Knops, Landsteiner-Wiener, Lutheran, MNS, Scianna and Yt blood groups.

54. The method of claim 53, wherein blood group loci to be amplified include at least one loci of the Rh blood groups.

55. A kit for genotyping a nucleic sample, the kit comprises:
a plurality of amplification primer pairs; wherein each amplification primer pair is configured to amplify at least a portion of a sequence of a blood group locus under amplification conditions,
wherein the blood groups are selected from the group consisting of: ABO, Colton, Cromer, Diego, Dombrock, Duffy, hemoglobin S, Kell, Kidd, Knops, Landsteiner-Wiener, Lutheran, MNS M/N, MNS S/s, Rh, Scianna, and Yt.

56. The kit of claim 55, wherein the kit further comprises a substrate having a plurality of sites, wherein each site contains one of the amplification primer pairs.

57. The kit of claim 55 or 56, wherein the kit further comprises at least one amplification primer pair configured to determine the copy number of a blood group locus.

58. The kit of any one of claims 55-57, wherein blood group loci to be amplified include at least one locus of each of the Colton, Cromer, Diego, Dombrock, Duffy, Kell, Kidd, Knops, Landsteiner-Wiener, Lutheran, MNS, Scianna and Yt blood groups.

59. The kit of claim 58, wherein blood group loci to be amplified include at least one loci of the Rh blood groups.

60. A genotyping reference nucleic acid molecule, the sequence of the nucleic acid molecule comprising: sequences of two or more blood group alleles, wherein each of the alleles is of a different blood group locus marker.

61. The genotyping reference nucleic acid molecule of claim 60, wherein the blood group locus markers are selected from the group consisting of ABO, Colton, Cromer, Diego, Dombrock, Duffy, hemoglobin S, Kell, Kidd, Knops, Landsteiner-Wiener, Lutheran, MNS, Rh, Scianna and Yt blood group.

62. The genotyping reference nucleic acid molecule of claim 60 or 61, wherein the sequences comprise at least one allele from each of RHD, RHCE, Cromer and Duffy.

63. The genotyping reference nucleic acid molecule of claim 62, wherein the alleles are the RHDΨ(wt), RHCE(c), Cromer(−), and Fy-a alleles.

64. The genotyping reference nucleic acid molecule of claim 62, wherein the alleles are the RHDΨ(duplicate), RHCE(C), Cromer(+), and Fy-b alleles.

65. A genotyping reference comprising:
at least two nucleic acid molecules, wherein each of the at least two nucleic acid molecules comprises a sequence of at least two different blood group alleles, and
wherein each of the at least two nucleic acid molecule contains only one allele sequence of a blood group locus marker.

66. The genotyping reference of claim 65, wherein the blood group loci are selected from the group consisting of ABO, Colton, Cromer, Diego, Dombrock, Duffy, hemoglobin S, Kell, Kidd, Knops, Landsteiner-Wiener, Lutheran, MNS, Rh, Scianna and Yt blood group.
